SEQUENCE LISTING (21 sequences)

SEQ ID NO: 1 - DNA, 1458 bp, Mus sp.
cgcgggcgccaccacggacgtgccacgcgggtggcccgagctattcggcagcactgaaggagccacccctcggccagggcgtgccaaggggttaaaatgaacgaagacccgaaggtcaatttaagcgggctgcctcgggactgtatagatgccggtgctccagagaacatctcagccgctgtcccctcccagggctctgttgcggagtcagaacccgagctcgttgtcaacccctgggacattgtcttgtgcagctcaggaaccctcatctgctgtgaaaatgccgttgtggtccttatcatcttccacagccccagcctgcgagcccccatgttcctactgataggcagcctggctcttgcagacctgctggctggcctgggactcatcatcaattttgtttttgcgtacctgcttcagtcagaagccaccaagctggtcaccatcggactcattgtcgcctctttctctgcctctgtctgcagtttgctggctattactgtggaccgctacctctcgctatattacgccctgacgtaccactccgagaggaccgtcacctttacctatgtcatgctagtgatgctctggggaacctccatctgcctggggctgctgcccgtcatgggctggaactgcttgagggacgagtccacctgcagcgtggtcagacctctcactaagaacaacgctgccatcctctccatctccttcctcttcatgtttgctctgatgcttcagctctacatccagatttgtaagattgtgatgaggcacgcccatcagatagccctgcagcaccacttcctggctacatcgcactatgtgactacccggaaaggggtctcgaccctggctctcatcctagggacctttgctgcctgctggatgcctttcaccctctattccttgatcgccgattacacctacccctcgatctatacctatgccaccctcctgcccgccacctacaattccatcatcaaccctgtcatttacgctttcagaaaccaagagatccagaaagccctctgcctcatttgctgtgggtgcatcccttcctcgctgtctcagagagctcggtctcccagcgatgtgtagcagccttctcctcataggacgctgcctctaccaagcgctcccacctcccagggcggccagtgatttccttccttaaattctttgcactggatctcacaagcagaagcaatgacatcttttagacacgtattgacagtggaaatcatcttaccagtgttttttaaaaaaaacaaaacaaaacaaaactcgacttctcggctcagcattctgttgtttggtttgggagttaggatttgtttgtttgtttgcttgtttgtttgtttggagggtgtaatgggacctcatgtggccatgaaattatacaaaagtctcgggattttttaacctaggcttgaaaataaatcaaagttttaaagg

SEQ ID NO: 2 - DNA, 1230 bp, Homo sapiens
aagcttgtggcatttggtactggtatctgagcaggggctggctttctgtttgtctgtgtgttttttgcatgatcttggattgtcaccctgctgtatttaaacattaaaaagcctgtcttttcgttgaagaggacaggggttaaaatgaatgaagacctgaaggtcaatttaagcgggctgcctcgggattatttagatgccgctgctgcggagaacatctcggctgctgtctcctcccgggttcctgccgtagagccagagcctgagctcgtagtcaacccctgggacattgtcttgtgtacctcgggaaccctcatctcctgtgaaaatgccattgtggtccttatcatcttccacaaccccagcctgcgagcacccatgttcctgctaataggcagcctggctcttgcagacctgctggccggcattggactcatcaccaattttgtttttgcctacctgcttcagtcagaagccaccaagctggtcacgatcggcctcattgtcgcctctttctctgcctctgtctgcagcttgctggctatcactgttgaccgctacctctcactgtactacgctctgacgtaccattcggagaggacggtcacgtttacctatgtcatgctcgtcatgctctgggggacctccatctgcctggggctgctgcccgtcatgggctggaactgcctccgagacgagtccacctgcagcgtggtcagaccgctcaccaagaacaacgcggccatcctctcggtgtccttcctcttcatgtttgcgctcatgcttcagctctacatccagatctgtaagattgtgatgaggcacgcccatcagatagccctgcagcaccacttcctggccacgtcgcactatgtgaccacccggaaaggggtctccaccctggctatcatcctggggacgtttgctgcttgctggatgcctttcaccctctattccttgatagcggattacacctacccctccatctatacctacgccaccctcctgcccgccacctacaattccatcatcaaccctgtcatatatgctttcagaaaccaagagatccagaaagcgctctgtctcatttgctgcggctgcatcccgtccagtctcgcccagagagcgcgctcgcccagtgatgtgtagcacccttgcacccaggaggactctgcatttaccaagcacttccactgcctggccaaggtttgagatgcttcccttgaattc

SEQ ID NO: 3 - DNA, 15 bp, Artificial Sequence
  Description of Artificial Sequence: Antisense oligonucleotide P0096
gggtcttcgttcatt

SEQ ID NO: 4 - DNA, 15 bp, Artificial Sequence
  Description of Artificial Sequence: Antisense oligonucleotide P0286
gataaggaccacaac

SEQ ID NO: 5 - DNA, 15 bp, Artificial Sequence
  Description of Artificial Sequence: Antisense oligonucleotide P0560
tgacataggtaaagg

SEQ ID NO: 6 - DNA, 15 bp, Artificial Sequence
  Description of Artificial Sequence: Antisense oligonucleotide P0805
cgatgtagccaggaa

SEQ ID NO: 7 - DNA, 15 bp, Artificial Sequence
  Description of Artificial Sequence:Antisense oligonucleotide P0991
gaaagcgtaaatgac

SEQ ID NO: 8 - DNA, 20 bp, Artificial Sequence
  Description of Artificial Sequence: ob-r Antisense oligonucleotide
gacacatcatctttcttcag

SEQ ID NO: 9 - DNA, 15 bp, Artificial Sequence
  Description of Artificial Sequence: Scrambled Antisense oligonucleotide
tggcgtcgtctttat

SEQ ID NO: 10 - skipped

SEQ ID NO: 11 - DNA, 2492 bp, Mus sp.
gcaagaagctgcaccggctttgcctgggggcggagcttgaggctggcagtgcccgtgaatcgtgaggggtctgggagcttcaggaaccaagtcatcgtgtctggtgggagggagctgactgctcttcttcttgacgtgtttccctcccctattcatgagcctattgagggcacaccttgaatcctgtgcttcccctattcacttatggtaagctttgatttacttctttgaccacgtgtggccaagaggtcttcaggacttagaggaatggaggggagggtgaagggacacagaaaagggaccaccgagaaaaagggacccctgtttagaggcctggaggcatcagaggacaaaaaccacaggacatgaattgggggtgtggggtgtgcaggacatccgtaccctacctgttatcctgacaggtttcttttctcgaagggcctcctgtcaggatacgggcctgccagcatctcataggacctttcttctacaggtaccatgatgtggggagcaggaagctctatggcctggttctcagctggctcaggcagtgtgaacgtgagcagcgtggacccagtagaggaacccacaggcccagctacactgctgccctctcccagggcctgggatgtggtgctgtgcatctcaggcaccctggtgtcctgcgagaacgcgctggtggtggccatcattgtgggcactcctgccttccgcgcccccatgttcctgctggtgggtagcttggccgtagcagacctgctggcaggcctgggcctggtcctgcactttgcggctgacttctgcattggctcaccagagatgagcttgatgctggtcggcgtgctagcaatggccttcactgccagcatcggcagcctgctggccattaccgttgaccgctacctttccctgtacaatgctctcacttactactcagagacaacggtaactcggacttatgtgatgctggccttggtgtgggtgggtgccctgggcctggggctggttcccgtgctggcctggaactgccgggatggcctgaccacgtgtggtgtggtctatccactctccaagaaccatctggtggttctggccatcgccttcttcatggtgtttggcatcatgttgcagctctatgcccagatctgccgcatcgtctgccgccatgcccagcagatcgccctccaacgacacctgctgcctgcctctcactacgtggccacccgcaagggcatcgccacattggctgtggtgcttggcgcctttgctgcctgttggttgcccttcactgtctactgcctcctgggagatgccgactctccccgtctctacacctatcttaccctgctcccagccacctacaactccatgatcaaccctgtcatctacgcctttcgcaaccaagatgtgcagaaggtgctgtgggccatctgctgctgctgttccacttccaagattccattccggtcccggtcccctagtgatgtctagtttcatcctggtgaacctgtggctttgactactatagaactccagagtgttaggtccaccagggcttaattccaatccccagctccacgcccttaagacccagctggttctggagttctaggacgttgggtgttttaggatcttattcaagattcctgcgggggcccaccccagctgaccgcgtggctctagaatgttccagatggtcagggtcccactcggaaatgtctcacagcccaagtggtttgcagttccagaaggctgggaattttacagtaccattccaagtccttgatcttccttcccagaaacttgaccttgatcatgtcgcttttgaatttcgaagctccagagtcagaggttaatcacttagttccctagatgagagaaagatttatctctatatatgtggacatacagagaaggtttatttatttatttattatttatttatttataaatttgcttatgggtggtaagggggaaaaaaagagacctacacctagaaatctagctcataccagggtatcctcagcctccatgaccccatttctgacctcagtttccgaagggggcgggatggaaaaagagaaaccacgtattttgttatttttgcttattttttatcagagaaatcatagaaaccagagcctcctccccaggcccgcactgctcgggtttgcagggagaacacaccagcctctggttttttatttttttaagaagccatcacctgagcaaccgagaattcctctgcgctgaggcccggcttccctctggtggccatttgggggaattgcaacccggcaaggcagtcgggattgtaaaatgcacccccatctctcactccagcctggctttcagcgccacagccatggcctgggggccaggccttaccctgaggtgcccgagaggaggcgggacaagagccagtacccccacccctctgccaatcgggttatggcccccagtgcattcctgttcagtccccaaccctactc

SEQ ID NO: 12 - DNA, 1982 bp, Homo sapiens
gaattctgggtttaggcgattctgggttagcaggcttgggcccaagtgctcgagtgctggggttgagttggatgacccggcgaagggtaagcattcctgcagggacccgaggccctggagggactgatggtcatctgaggttaagccggcaggaggcgtctggggcagggacccaggggtccgaacagcaggaggtgtccaatttaggaatccaggtatccaggcaggaggcagagtcaggcccaaaatccaggtgtccgggcagaaggaggcatctgactcagggatccagtcttccagcaggggggtctgcccagagacctagagattcaggcggaaagaagggtccagccaggaacagaagtctgggagggtggggggtggggggctgagttcccaggtgttaccgcgggagtgcggggtgatggctggcgggagcttcacgcgcattccctgggggcggagtctaaggctggcagtactgaaaatcgtgaggggtctgggagcctcaggcacgagctagcatcatcatctctgatgggagggagcggagctgctcagtctccctgatggggtagatgtttccctaccccaatcttgagccttcgggggcatcccctggatcctatggccctcttccacttatagtaccccttcttctgtctctcctctggtcacacacgtgggaaaaagagacctgcaggacctaggacagggatccccaggaagagacccctgtttagaggcctgggggcattggaggggacagcggtatcctgggaagagccccagggcatgaatgtggggataaggcattgggactctatcaggtatcctgaggagagactcccaccacgtatcctgagaagcacctcaccccctccagaccccaactcccatcacccagcttggtcagcttctcacaaggcctttctcctgcaggtaccatgatgtggggtgcaggcagccctctggcctggctctcagctggctcaggcaacgtgaatgtaagcagcgtgggcccagcagaggggcccacaggtccagccgcaccactgccctcgcctaaggcctgggatgtggtgctctgcatctcaggcaccctggtgtcctgcgagaatgcgctagtggtggccatcatcgtgggcactcctgccttccgtgcccccatgttcctgctggtgggcagcctggccgtggcagacctgctggcaggcctgggcctggtcctgcactttgctgctgtcttctgcatcggctcagcggagatgagcctggtgctggttggcgtgctggcaatggcctttaccgccagcatcggcagtctactggccatcactgtcgaccgctacctttctctgtacaatgccctcacctactattcagagacaacagtgacacggacctatgtgatgctggccttagtgtggggaggtgccctgggcctggggctgctgcctgtgctggcctggaactgcctggatggcctgaccacatgtggcgtggtttatccactctccaagaaccatctggtagttctggccattgccttcttcatggtgtttggcatcatgctgcagctctacgcccaaatctgccgcatcgtctgccgccatgcccagcagattgcccttcagcggcacctgctgcctgcctcccactatgtggccacccgcaagggcattgccacactggccgtggtgcttggagcctttgccgcctgctggttgcccttcactgtctactgcctgctgggtgatgcccactctccacctctctacacctatcttaccttgctccctgccacctacaactccatgatcaaccctatcatctacgccttccgcaaccaggatgtgcagaaagtgctgtgggctgtctgctgctgctgttcctcttccaagatccccttccgatcccgctcccccagtgatgtctagctgagtcttcatgacccttcaaccctgattactacagaattc

SEQ ID NO: 13 - DNA, 15 bp, Artificial Sequence
  Description of Artificial Sequence: Antisense oligonucleotide P0519
aaccaggccatagag

SEQ ID NO: 14 - DNA, 15 bp, Artificial Sequence
  Description of Artificial Sequence: Antisense oligonucleotide P0973
acccacccacaccaa

SEQ ID NO: 15 - DNA, 15 bp, Artificial Sequence
  Description of Artificial Sequence: Antisense oligonucleotide P1080
gccagaaccaccaga

SEQ ID NO: 16 - DNA, 15 bp, Artificial Sequence
  Description of Artificial Sequence: Antisense oligonucleotide P1332
agggtaagataggtg

SEQ ID NO: 17 - DNA, 15 bp, Artificial Sequence
  Description of Artificial Sequence: Antisense oligonucleotide P1478
actagacatcactag

SEQ ID NO: 18 - skipped

SEQ ID NO: 19 - DNA, 15 bp, Artificial Sequence
  Description of Artificial Sequence: “Sense” oligonucleotide (complement of Antisense oligonucleotide P1080)
tctggtggttctggc

SEQ ID NO: 20 - DNA, 1230 bp, Homo sapiens
gaattcaagggaagcatctcaaaccttggccaggcagtggaagtgcttggtaaatgcagagtcctcctgggtgcaagggtgctacacatcactgggcgagcgcgctctctgggcgagactggacgggatgcagccgcagcaaatgagacagagcgctttctggatctcttggtttctgaaagcatatatgacagggttgatgatggaattgtaggtggcgggcaggagggtggcgtaggtatagatggaggggtaggtgtaatccgctatcaaggaatagagggtgaaaggcatccagcaagcagcaaacgtccccaggatgatagccagggtggagacccctttccgggtggtcacatagtgcgacgtggccaggaagtggtgctgcagggctatctgatgggcgtgcctcatcacaatcttacagatctggatgtagagctgaagcatgagcgcaaacatgaagaggaaggacaccgagaggatggccgcgttgttcttggtgagcggtctgaccacgctgcaggtggactcgtctcggaggcagttccagcccatgacgggcagcagccccaggcagatggaggtcccccagagcatgacgagcatgacataggtaaacgtgaccgtcctctccgaatggtacgtcagagcgtagtacagtgagaggtagcggtcaacagtgatagccagcaagctgcagacagaggcagagaaagaggcgacaatgaggccgatcgtgaccagcttggtggcttctgactgaagcaggtaggcaaaaacaaaattggtgatgagtccaatgccggccagcaggtctgcaagagccaggctgcctattagcaggaacatgggtgctcgcaggctggggttgtggaagatgataaggaccacaatggcattttcacaggagatgagggttcccgaggtacacaagacaatgtcccaggggttgactacgagctcaggctctggctctacggcaggaacccgggaggagacagcagccgagatgttctccgcagcagcggcatctaaataatcccgaggcagcccgcttaaattgaccttcaggtcttcattcattttaacccctgtcctcttcaacgaaaagacaggctttttaatgtttaaatacagcagggtgacaatccaagatcatgcaaaaaacacacagacaaacagaaagccagcccctgctcagataccagtaccaaatgccacaagctt

SEQ ID NO: 21 - DNA, 1982 bp, Homo sapiens
gaattctgtagtaatcagggttgaagggtcatgaagactcagctagacatcactgggggagcgggatcggaaggggatcttggaagaggaacagcagcagcagacagcccacagcactttctgcacatcctggttgcggaaggcgtagatgatagggttgatcatggagttgtaggtggcagggagcaaggtaagataggtgtagagaggtggagagtgggcatcacccagcaggcagtagacagtgaagggcaaccagcaggcggcaaaggctccaagcaccacggccagtgtggcaatgcccttgcgggtggccacatagtgggaggcaggcagcaggtgccgctgaagggcaatctgctgggcatggcggcagacgatgcggcagatttgggcgtagagctgcagcatgatgccaaacaccatgaagaaggcaatggccagaactaccagatggttcttggagagtggataaaccacgccacatgtggtcaggccatccaggcagttccaggccagcacaggcagcagccccaggcccagggcacctccccacactaaggccagcatcacataggtccgtgtcactgttgtctctgaatagtaggtgagggcattgtacagagaaaggtagcggtcgacagtgatggccagtagactgccgatgctggcggtaaaggccattgccagcacgccaaccagcaccaggctcatctccgctgagccgatgcagaagacagcagcaaagtgcaggaccaggcccaggcctgccagcaggtctgccacggccaggctgcccaccagcaggaacatgggggcacggaaggcaggagtgcccacgatgatggccaccactagcgcattctcgcaggacaccagggtgcctgagatgcagagcaccacatcccaggccttaggcgagggcagtggtgcggctggacctgtgggcccctctgctgggcccacgctgcttacattcacgttgcctgagccagctgagagccaggccagagggctgcctgcaccccacatcatggtacctgcaggagaaaggccttgtgagaagctgaccaagctgggtgatgggagttggggtctggagggggtgaggtgcttctcaggatacgtggtgggagtctctcctcaggatacctgatagagtcccaatgccttatccccacattcatgccctggggctcttcccaggataccgctgtcccctccaatgcccccaggcctctaaacaggggtctcttcctggggatccctgtcctaggtcctgcaggtctctttttcccacgtgtgtgaccagaggagagacagaagaaggggtactataagtggaagagggccataggatccaggggatgcccccgaaggctcaagattggggtagggaaacatctaccccatcagggagactgagcagctccgctccctcccatcagagatgatgatgctagctcgtgcctgaggctcccagacccctcacgattttcagtactgccagccttagactccgcccccagggaatgcgcgtgaagctcccgccagccatcaccccgcactcccgcggtaacacctgggaactcagccccccaccccccaccctcccagacttctgttcctggctggacccttctttccgcctgaatctctaggtctctgggcagacccccctgctggaagactggatccctgagtcagatgcctccttctgcccggacacctggattttgggcctgactctgcctcctgcctggatacctggattcctaaattggacacctcctgctgttcggacccctgggtccctgccccagacgcctcctgccggcttaacctcagatgaccatcagtccctccagggcctcgggtccctgcaggaatgcttacccttcgccgggtcatccaactcaaccccagcactcgagcacttgggcccaagcctgctaacccagaatcgcctaaacccagaattc

(19) United States
(12) Patent Application Publication
(10) Pub. No.: US 2003/0096785 A1
(43) Pub. Date: May 22, 2003

(54) Control of hunger, satiety, and food intake by modulating expression and activity of cephalic G protein-coupled receptors

(75) Inventors: Alain Stricker-Krongrad, Cambridge, MA (US); Wei Gu, Cambridge, MA (US)

Correspondence Address: MILLENNIUM PHARMACEUTICALS, INC., 75 Sidney Street, Cambridge, MA, 02139, US

(73) Assignee: Millennium Pharmaceuticals, Inc.

(21) Appl. No.: 10/287,224

(22) Filed: Nov. 4, 2002

Related U.S. Application Data
(63) Continuation of application No. 09/661,760, filed on Sep. 14, 2000, now abandoned.

(51) Int. Cl. A61K 48/00

(52) U.S. Cl. 514/44

(57) ABSTRACT

The invention relates to the discovery that two human proteins (G protein-coupled receptors designated GPR12 and GPR3) and their mammalian orthologs, which are known to be expressed in various brain tissues including in the hypothalamus, are involved in regulation of food intake in mammals. The invention includes compositions and methods for modulating expression and activity of these proteins, and methods for identifying such compositions. The invention also includes methods of treating disorders such as obesity, diabetes, and inanity, and methods of modulating body weight.

### CROSS-REFERENCE TO RELATED APPLICATIONS

Not Applicable.

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

A diverse array of extracellular conditions can induce transduction of signals in one or more cellular signaling systems which involve guanine nucleotide-binding regulatory proteins (G proteins). Among the components of these signaling systems are several families of integral membrane proteins designated G protein-coupled receptors (GPCRs). By way of example, GPCRs are believed to be involved in intracellular signaling relating to perception of light, temperature, and compounds such as odorants, ions, amino acids, peptides and polypeptides, nucleotides, and nucleic acids. GPCRs are expressed in a variety of tissues, including brain tissues of various types, and are believed to have numerous physiological roles, such as modulation of mood, motor activity, nociception, thermoregulation, learning, and other behaviors.

GPCRs are membrane-spanning proteins which exhibit several common structural and sequence features. For instance, many GPCRs have seven transmembrane domains, bind with their ligand in a conserved extracellular hydrophobic pocket, and bind with a G protein by means of the intracellular portions (often including the third intracellular loop and the intracellular carboxyl-terminal domain). Following interaction of a GPCR with its extracellular ligand, conformation or other changes in the GPCR render it able to bind with an intracellular G protein. Interaction between the GPCR and the G protein can lead to alteration of the G protein such that the G protein can induce or catalyze further intracellular changes. In this way, presence of an extracellular ligand of a GPCR can lead to changes in the metabolism or the structure of a cell.

Among the GPCRs that have been identified by others are human GPCRs designated hGPR12 (see, e.g., Song et al., 1995, Genomics 28:347-349; GENBANK™ accession number U18548) and hGPR03 (see, e.g., Marchese et al., 1994, Genomics 23:609-618; Iismaa et al., 1994, Genomics 24:391-394; Song et al., supra; Eggerickx et al., 1995, Biochem. J. 309:837-843; GENBANK™ accession numbers X83956, AL096774, L32831, U18550, and U13668) and their murine orthologs, designated mGPRO1 (see, e.g., Saeki et al., 1993, FEBS Lett. 336:371-322) and mGPR2t (see, e.g., Saeki et al., supra), respectively. The rat ortholog (designated R334) of hGPR12 and mGPR01 has also been described (see, e.g., Eidne et al., 1991, FEBS Lett. 292:243-248). The high levels of amino acid sequence identity which exists among hGPR12, mGPR01, and R334 and between hGPR03 and mGPR21 is understood to indicate that these groups of proteins have common ligands and exhibit common physiological activities (see, e.g., Eggerickx et al., supra; lismaa et al., supra).

Expression of the rat ortholog of hGPR12 has been detected in brain, pituitary gland, and testis tissues (Eidne et al., supra). Expression of mGPR21 has been detected in brain (including cerebellum, frontal cortex, thalamus, and hindbrain), brainstem, spinal cord, eye, testis, and ovary tissues (Eggerickx et al., supra; Saeki et al., supra). Within brain tissue, mGPR21 was found to be expressed prominently in medial habenular nucleus, cerebral cortex, hippocampus, olfactory bulb, and striatum portions of the brain. Expression of mGPR01 has been detected in brain structures including hippocampus (including granular cells of the dentate gyrus and CA2-3), amygdaloid nuclei, piriform cortex, olfactory bulb (both external granular lamina and mitral cellular lamina), thalamus, striatum, ventromedial hypothalamic nuclei, and cerebral cortex (Saeki et al., supra). Although the structure and anatomical location of these receptors are known, the physiological functions of these receptors has not previously been understood.

The present invention relates to functions which have, in the experiments described herein, been associated with expression and activity of these receptors.

### BRIEF SUMMARY OF THE INVENTION

The invention relates to a composition for suppressing hunger in a mammal (e.g., a human). The composition comprises an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03. The polynucleotide can, for example, have the nucleotide sequence of either of SEQ ID NOs: 2 and 12. The oligonucleotide can have a nucleotide sequence selected from the group consisting of SEQ ID NOs: 3-7 and 13-17, and in one embodiment comprises 15 nucleotide residues.

The composition can further comprises a pharmaceutically acceptable carrier, and can, for example, be formulated for intrathecal administration, for sustained release of the oligonucleotide, or both.

The invention also relates to a method of suppressing hunger in a mammal (e.g., a human). This method comprises intrathecally administering to the mammal an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03.

In another aspect, the invention relates to a composition for enhancing satiety in a mammal such as a human. The composition comprises an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03.

The invention further relates to a method of enhancing satiety in a mammal (e.g., a human). This method comprises intrathecally administering to the mammal an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03.

In yet another aspect, the invention relates to a method of assessing whether a composition is a modulator of hunger in a mammal. This method comprises comparing

a) the change in the cytoplasmic concentration of a signal transduction mediator selected from the group consisting of Ca²⁺ ion, cyclic AMP, inositol 1,4,5-triphosphate, and 1,2-diacylglycerol attributable to the presence of the composition in a medium contacting a first cell which comprises a G protein-coupled receptor protein selected from the group consisting of hGPR12, hGPR03, the mammal's ortholog of hGPR12, and the mammal's ortholog of hGPR03; and

b) the change in the cytoplasmic concentration of the same signal transduction mediator attributable to the presence of the composition in a medium contacting a second cell of the same type which does not comprise the G protein-coupled receptor protein.

A difference between

i) the change attributable to the presence of the composition in a medium contacting the first cell and

ii) the change attributable to the presence of the composition in a medium contacting the second cell

is an indication that the composition is a modulator of hunger in the mammal.

The invention also includes a method of assessing whether a composition is a modulator of satiety in a mammal. This method comprises comparing

a) the change in the cytoplasmic concentration of a signal transduction mediator selected from the group consisting of Ca2+ ion, cyclic AMP, inositol 1,4,5-triphosphate, and 1,2-diacylglycerol attributable to the presence of the composition in a medium contacting a first cell which comprises a G protein-coupled receptor protein selected from the group consisting of hGPR12, hGPR03, the mammal's ortholog of hGPR12, and the mammal's ortholog of hGPR03; and

b) the change in the cytoplasmic concentration of the same signal transduction mediator attributable to the presence of the composition in a medium contacting a second cell of the same type which does not comprise the G protein-coupled receptor protein.

A difference between

i) the change attributable to the presence of the composition in a medium contacting the first cell and

ii) the change attributable to the presence of the composition in a medium contacting the second cell

is an indication that the composition is a modulator of hunger in the mammal.

In still another aspect, the invention includes an antisense oligonucleotide consisting of from 15 to 300 nucleotide residues. The nucleotide sequence of the oligonucleotide is at least 75% identical with a portion of one of SEQ ID NOs: 20 and 21, as assessed using the NBLAST program with score=100, wordlength=12, gap opening penalty=11, gap extension penalty=11, and lambda ratio=0.85. Alternatively, the nucleotide sequence of the oligonucleotide is at least 75% identical with a portion of one of SEQ ID NOs: 20 and 21, as assessed using the ALIGN program with PAM120 weight residue table, gap opening penalty=11, gap extension penalty=1, and lambda ratio=0.85. In one embodiment, the nucleotide sequence of the oligonucleotide is at least 75% identical with a portion of one of SEQ ID NOs: 20 and 21, as assessed by non-gapped sequence alignment.

The invention further relates to a method of modulating food intake in a mammal (e.g., a human). This method comprises intrathecally administering to the mammal an antibody raised against an extracellular portion of a protein selected from the group consisting of hGPR12, hGPR03, the mammal's ortholog of hGPR12, and the mammal's ortholog of hGPR03.

The invention also includes a method of making an antibody for modulating food intake in a mammal. The method comprises

administering to an immunocompetent vertebrate a membrane-embedded protein selected from the group consisting of hGPR12, hGPR03, the mammal's ortholog of hGPR12, and the mammal's ortholog of hGPR03; and thereafter

recovering an antibody which binds specifically with the protein from the vertebrate's serum. The recovered antibody is useful for modulating food intake in a mammal.

The invention further includes a method of making an antibody for modulating food intake in a mammal. This method comprising

administering to an immunocompetent vertebrate a membrane-embedded protein selected from the group consisting of hGPR12, hGPR03, the mammal's ortholog of hGPR12, and the mammal's ortholog of hGPR03; thereafter

isolating an antibody-producing cell which produces an antibody which binds specifically with the protein from the vertebrate;

forming a hybridoma using the antibody-producing cell; and

recovering the antibody from a medium in which the hybridoma is maintained. The recovered antibody is useful for modulating food intake in a mammal.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a graph that depicts the effects of intracerebroventricular (ICV) administration of a single 0.65 nanomole dose of selected anti-mGPR01 ASOs on cumulative food intake (CFI) in mice.

FIG. 2 is a graph that depicts the effects of ICV administration of a single 0.65 nanomole dose of selected anti-mGPROI ASOs on CFI in mice following an enforced fast.

FIG. 3 is a graph that depicts the effects of ICV administration of a selected single dose of anti-mGPR01 ASO P0096 on CFI in mice.

FIG. 4 is a graph that depicts the effects of ICV administration of a selected single dose of anti-mGPR01 ASO P0096 on CFI in mice following an enforced fast.

FIG. 5 is a graph that depicts the effects of ICV administration of a single dose of PBS, anti-mGPR01 ASO P0096, or an oligonucleotide designated “Scramble” (i.e., a control oligonucleotide consisting of the same nucleotide residues, but in a scrambled sequence) on CFI in mice which were permitted to feed at will (+/+) and in mice which were forced to fast for 24 hours and then permitted to feed at will (−/+).

FIG. 6 is a graph that depicts the effects of intracerebroventricular (ICV) administration of a single 0.65 nanomole dose of selected anti-mGPR21 ASOs on cumulative food intake (CFI) in mice.

FIG. 7 is a graph that depicts the effects of ICV administration of a single 0.65 nanomole dose of selected anti-mGPR21 ASOs on CFI in mice following an enforced fast.

FIG. 8 is a graph that depicts the effects of ICV administration of a selected single dose of anti-mGPR21 ASO P1080 on CFI in mice.

FIG. 9 is a graph that depicts the effects of ICV administration of a selected single dose of anti-mGPR21 ASO P1080 on CFI in mice following an enforced fast.

FIG. 10 is a graph that depicts the effects of ICV administration of a single dose of PBS, anti-mGPR01 ASO P1080, or an oligonucleotide designated “Sense” on CFI in mice which were permitted to feed at will (+/+) and in mice which were forced to fast for 24 hours and then permitted to feed at will (−/+).

FIG. 11, comprising FIGS. 11A and 11B, lists the nucleotide sequence of the sense (FIG. 11A; SEQ ID NO: 2) and antisense (FIG. 11B; SEQ ID NO: 20) strands of a cDNA encoding hGPR12.

FIG. 12, comprising FIGS. 12A through 12D, lists the nucleotide sequence of the sense (FIGS. 12A and 12B; SEQ ID NO: 12) and antisense (FIGS. 12C and 12D; SEQ ID NO: 21) strands of a cDNA encoding hGPR03.

FIG. 13 lists the nucleotide sequence of the sense strand of a cDNA encoding mGPR01 (SEQ ID NO: 1).

FIG. 14, comprising FIGS. 14A and 14B, lists the nucleotide sequence of the sense strand of a cDNA encoding mGPR21 (SEQ ID NO: 11).

### DETAILED DESCRIPTION OF THE INVENTION

The invention is based on the discovery that two previously identified G protein-coupled receptors (GPCRs) are involved in physiological regulation of satiety, hunger, and food intake in mammals. One of the GPCRs is designated hGPR12 in humans, mGPR01 (or, alternatively, mGPR1) in mice, and R334 in rats. The other GPCR is designated hGPR03 (or, alternatively, hGPR3) in humans and mGPR21 in mice. It has also been discovered that antisense oligonucleotides (ASOs) derived from the DNA sequences encoding these GPCRs can be used to modulate satiety and hunger in mammals.

The invention includes compounds (e.g., ASOs) which can be used to interfere with normal expression and physiological functioning of GPCRs of the hGPR12 family (i.e., including mGPR01 and R334) and the hGPR03 family (i.e., including mGPR21). Such compounds can be used to modulate satiety, hunger, and food intake in mammals, and thus can be used to treat disorders such as obesity, inanity, and diabetes and to effect body weight loss or gain in an individual mammal. Such compounds can also be used as appetite suppressants (e.g., in patients needing or wishing to decrease body weight) or enhancers (e.g., in patients experiencing eating disorders or receiving appetite-inhibiting medications). The invention also includes methods of using such compounds to treat (i.e., alleviate, inhibit, or prevent) these conditions and methods of identifying such compounds.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the coding strand of an mRNA molecule which is normally expressed in a cell and which encodes a protein. It is not necessary that the antisense sequence be complementary solely to the coding portion of the coding strand of the mRNA molecule. The antisense sequence may be complementary to one or more regulatory sequences (e.g. a ribosome binding site, Shine-Dalgarno sequence, translation initiation codon, splicing junction, or translation termination codon) specified on the coding strand of an mRNA molecule encoding a protein, which regulatory sequences modulate expression of the coding sequences.

“Intrathecal” administration of a composition refers to delivery of the composition to the cerebrospinal fluid, i.e. to the interior of the dural sheath enclosing the brain and spinal cord, including delivery of the composition into a cerebral ventricle.

“Intracerebroventricular” (ICV) administration of a composition refers to delivery of the composition into a cerebral ventricle.

“Subdural” administration of a composition refers to deliver of the composition between the interior surface of the dural sheath and the exterior surface of the brain or spinal cord.

As used herein, the term “hybridizes under stringent conditions” is used to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, New York (1989), 6.3.1-6.3.6. A preferred, non-limiting example of stringent hybridization conditions is hybridization in 6×sodium chloride/sodium citrate (SSC) at about 45° C., followed by one or more washes in 0.2×SSC, 0.1% (w/v) sodium dodecyl sulfate at 65° C. Polynucleotides which remain annealed under these conditions hybridize under stringent conditions.

As used herein the term “pharmaceutically acceptable carrier” includes solvents, dispersion media, coatings, anti-bacterial and anti-fungal agents, isotonic and absorption delaying agents, and the like, compatible with pharmaceutical administration. The use of such media and agents for pharmaceutically active substances is well known in the art.

Description

Modulation of Food Intake and Body Weight

Sensation of hunger leads mammals to ingest food. Hunger sensation includes objective sensations such as stomach contractions or even stomach pain. Sensation of hunger also includes psychic sensations of hunger, which are experienced even by those whose stomachs have been surgically removed. Once a mammal has begun eating, it will often continue to eat until it experiences satiety or ‘fullness,’ at which point the mammal's appetite is suppressed, and ingestion slows or stops.

It is known that the lateral nuclei of the hypothalamus are involved in stimulation of eating in mammals, and that the ventromedial nuclei of the hypothalamus are involved in sensation of satiety. Other portions of the brain are also known to be involved in sensation of hunger and satiety and control of the eating impulse and the nutritional status of mammals. For example, the paraventricular nuclei, the dorsomedial nuclei of the hypothalamus, the area postrema of the lower brain stem, the caudal medial nucleus of the solitary tract, the amygdala, and the prefrontal cortex have all been implicated in various aspects of regulation of food intake. Furthermore, psychological stress is known to influence eating behavior. The physiological and biochemical bases for regulation of food intake and body weight are poorly understood.

The present inventors have discovered that two human proteins known to be expressed in various brain tissues, including in the hypothalamus, are involved in regulation of food intake in mammals. Regulating expression or activity (i.e., ligand- or G protein-binding activity) of these proteins, or their orthologs in other mammals, can modulate satiety and hunger in mammals, thereby regulating food intake and, over time, body weight.

The inventors have discovered that the GPCR designated hGPR12, its murine ortholog designated mGPR01, and its rat ortholog designated R334, modulates food intake. Modulating expression of hGPR12 in brain tissue or the ability of hGPR12 to interact with its physiological ligand (which remains unknown) can lead to enhancement or suppression of hunger and can also lead to enhancement or suppression of satiety. Examples of ASOs which can be used to inhibit expression of hGPR12, mGPR01, or R334 are described herein. When such ASOs are administered intrathecally to a mammal (e.g., by intracerebroventricular {ICV} injection), hunger is suppressed, satiety is enhanced, or both, and the mammal consumes less food. Consequently, the body weight of the mammal decreases over time as food consumption remains depressed while metabolism continues.

The inventors have also discovered that the GPCR designated hGPR03, and its munne ortholog designated mGPR21, modulates food intake. Examples of ASOs which can be used to inhibit expression of hGPR03 or mGPR21 are described herein. As with the hGPR12 family of GPCRs, when such ASOs are administered intrathecally to a mammal (e.g., by ICV injection), hunger is suppressed, satiety is enhanced, or both, and the mammal consumes less food. Consequently, the body weight of the mammal decreases over time as food consumption remains depressed while metabolism continues.

GPCRs are known to bind one or more ligands (or otherwise sense a stimulus) at the exterior (i.e., non-cytoplasmic) surface of the cell, whereupon a conformational or other change in the GPCR alters its interaction with an intracellular guanine nucleotide-binding protein (G protein) at the cytoplasmic face of the GPCR. This altered interaction induces a change in the G protein which can lead to alteration of the interaction between the G protein and one or more other cellular components, thereby transmitting the signal represented by the presence of the extracellular ligand to intracellular processes, whereby physiological changes (e.g., hormone production) in the cell can be effected. Thus, interference with binding of a physiological ligand with a GPCR of the hGPR12 or hGPR03 family can alter the signal transduction ordinarily facilitated by those proteins. Because these proteins are involved in regulation of hunger and satiety, compounds which interfere with binding between these proteins and their physiological ligands can be used to modulate food intake and body weight.

Antibodies

Examples of compounds which can be used to interfere with GPCR-ligand binding include antibodies which bind specifically with the portion of the GPCR which is normally present on the exterior surface of a cell which expresses the GPCR. Such antibodies can be made using conventional methods. For example, such antibodies can be generated by administering a cell which expresses the GPCR (or a membrane vesicle comprising the GPCR in its physiological orientation) to an immunocompetent vertebrate (e.g., a mouse, rat, rabbit, sheep, goat, or kangaroo), and subsequently obtaining serum containing the antibodies, or antibody-producing cells which produce the antibodies, from the vertebrate. Thus, antibodies which bind specifically with one or more exterior (i.e., extracellular) portions of a GPCR of the hGPR12 or hGPR03 families can be used to regulate food intake and body weight in mammals.

Accordingly, antibodies which can be used in the method described herein can be ones which specifically bind with an extracellular portion of hGPR12, hGPR03, or a mammalian ortholog of either of these. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds with a given polypeptide is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against (i.e., which bind specifically with) one or more extracellular portions of a protein of the hGPR12 or hGPR03 families. Particularly preferred polyclonal antibody preparations are ones that contain only antibodies directed against one protein of these families.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (Eds.) John Wiley & Sons, Inc., New York, N.Y.). Hybridoma cells producing a monoclonal antibody of interest are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a GPCR described herein can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SURFZAP™ Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619, WO 91/17271, WO 92/20791, WO 92/15679, WO 93/01288, WO 92/01047, WO 92/09690, and WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Recombinant antibodies, such as chimeric and humanized monoclonal antibodies, comprising both human and non-human portions, which can be made using standard recombinant DNA techniques, are within the scope of the invention. A chimeric antibody is a molecule in which different portions of the antibody amino acid sequence are derived from different animal species, such as those having a variable region derived from a murine monoclonal antibody and a constant region derived from a human immunoglobulin. (See, e.g., Cabilly et al., U.S. Pat. No. 4,816,567; and Boss et al., U.S. Pat. No. 4,816,397). Humanized antibodies are antibody molecules which are obtained from non-human species, which have one or more complementarity-determining regions (CDRs) derived from the non-human species, and which have a framework region derived from a human immunoglobulin molecule. (See, e.g., Queen, U.S. Pat. No. 5,585,089). Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. (1994) Bio/technology 12:899-903).

An antibody directed against a GPCR of the hGPR12 or hGPR03 families (e.g., a monoclonal antibody) can be used to identify regions of the brain or other body structures which are involved in modulating food intake. Such antibodies can also be used to monitor protein levels in tissue (e.g., on ventricular hypothalmic surfaces) as part of a clinical testing procedure, e.g., to determine the efficacy of a treatment regimen intended to reduce expression of the protein thereon. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An antibody (or fragment thereof) can be conjugated to a therapeutic moiety such as a cytotoxin, a therapeutic agent, or a radioactive agent (e.g., a radioactive metal ion). Cytotoxins and cytotoxic agents include any agent that is detrimental to cells. Examples of such agents include radionuclides, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin {formerly designated daunomycin} and doxorubicin), antibiotics (e.g., dactinomycin {formerly designated actinomycin}, bleomycin, mithramycin, and anthramycin), and anti-mitotic agents (e.g., vincristine and vinblastine). Antibodies conjugated with a therapeutic or cytotoxic substance can be used to deliver the substance to cells and tissues which are involved in modulation of food intake (e.g., in order to ablate a portion of such tissue).

Techniques for conjugating a therapeutic moiety to an antibody are well known (see, e.g., Arnon et al., 1985, “Monoclonal Antibodies For Immunotargeting Of Drugs In Cancer Therapy”, in Monoclonal Antibodies And Cancer Therapy, Reisfeld et al., Eds., Alan R. Liss, Inc. pp. 243-256; Hellstrom et al., 1987, “Antibodies For Drug Delivery”, in Controlled Drug Delivery, 2nd ed., Robinson et al., Eds., Marcel Dekker, Inc., pp. 623-653; Thorpe, 1985, “Antibody Carriers Of Cytotoxic Agents In Cancer Therapy: A Review”, in Monoclonal Antibodies '84: Biological And Clinical Applications, Pinchera et al., Eds., pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al., Eds., Academic Press, pp. 303-316, 1985; and Thorpe et al., 1982, Immunol. Rev., 62:119-158). Alternatively, an antibody can be conjugated to a second antibody to form an antibody heteroconjugate as described by Segal in U.S. Pat. No. 4,676,980.

Antisense Oligonucleotides (ASOs)

In one aspect, the invention relates to ASOs which can be used to inhibit expression of a GPCR of the hGPR12 family or of the hGPR03 family (i.e., to inhibit expression of any of hGPR12, hGPR03, mGPR01, mGPR21, and R334). Inhibition of the GPCR inhibits (i.e., suppresses, reduces, or eliminates) hunger, enhances (i.e., generates or increases) satiety, or both. Design of ASOs is a conventional procedure for genes (e.g., the genes of the hGPR12 and hGPR03 families) having known sequences.

ASOs can have a size from 15 to 300 or more nucleotide residues (preferably in the ranges 15-200, 15-100, 20-50, 20-35, 25-50, or 25-35 nucleotide residues) and have a sequence that is either exactly complementary to the sense strand of the gene or exhibits high homology with a polynucleotide that is exactly complementary to the corresponding portion of the sense strand. For example, useful ASOs can have a sequence that is 75%, 80%, 85%, 90%, 95%, or 98% or more homologous with a polynucleotide that is exactly complementary to the corresponding of the sense strand. Such homology can be assessed by alignment using a non-gapped method (i.e., a method in which introduction of gaps into the sequences being aligned is not permitted). Alternatively, homology can be assessed using algorithms described in the art. Two examples of such algorithms are embodied in the NBLAST program (Altschul et al., 1997, Nucleic Acids Res. 25:3389-3402; parameters: score=100 and wordlength=12) and the ALIGN program (version 2.0; Myers and Miller, 1988, CABIOS 4:11-17; parameters: PAM120 weight residue table, gap existence penalty=12, and gap extension penalty=4). ASOs are preferably designed to be complementary to a regulatory sequence (e.g., a Shine-Dalgarno sequence or translation initiation codon) of the sense strand.

Methods of generating antisense oligonucleotides which can be used to inhibit (or in certain instances to enhance) expression of a gene are known in the art, and include, for example, methods such as ‘gene walking.’ In gene walking methods, a variety of ASOs are prepared, each having a sequence completely, or at least highly, complementary to a portion of the sense strand of the gene. Expression of the gene in the presence and absence of each ASO is compared in order to determine the effect of the ASO on expression. ASOs having the desired type and degree of effect are selected or, alternately, used to identify regions of the gene from which more efficacious ASOs can be derived. Thus, ASO design methods can include numerous iterations or screening stages.

ASOs for inhibiting expression of GPCRs of the hGPR12 and hGPR03 families are preferably designed such that they hybridize under stringent conditions with a portion of the sense strand of a cDNA encoding such a GPCR. By way of example, a useful ASO can be designed such that it hybridizes under stringent conditions with a cDNA encoding one of hGPR12 (e.g., a cDNA having the sequence SEQ ID NO: 2), hGPR03 (e.g., a cDNA having the sequence SEQ ID NO: 12), mGPR01 (e.g., a cDNA having the sequence SEQ ID NO: 1), mGPR21 (e.g., a cDNA having the sequence SEQ ID NO: 11), and R334. ASOs can also be designed such that they exhibit high homology (e.g., 75%, 80%, 85%, 90%, 95%, or 98% or higher, as assessed using the methods described herein) with the antisense strands of a cDNA encoding a GPCR of the hGPR12 or hGPR03 family (e.g. a DNA strand having the nucleotide sequence SEQ ID NO: 20 or 21).

An ASO can be, for example, about 15, 20, 25, 30, 35, 40, 45, or 50 or more nucleotides in length. An ASO of the invention can be constructed using chemical synthesis and enzymatic ligation reactions using procedures known in the art. For example, an ASO can be chemically synthesized using naturally occurring nucleotides or variously modified nucleotides designed to increase the biological stability of the molecules or to increase the physical stability of the duplex formed between the ASO and its target nucleic acid. Examples of modified nucleotides include phosphorothioate derivatives and acridine substituted nucleotides. Other examples of modified nucleotides which can be used to generate ASOs include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methyl guanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine. Alternatively, an ASO can be produced biologically using an expression vector into which a nucleic acid has been sub-cloned in an antisense orientation (i.e., RNA transcribed from the inserted nucleic acid will be of an antisense orientation to the target nucleic acid, described further in the following subsection).

An ASO described herein can be administered to a subject or generated in situ such that it hybridizes with (i.e., binds with) RNA or DNA in the cytoplasm or nucleus of a cell. Binding of the ASO with RNA or DNA encoding a GPCR of the hGPR12 or hGPR03 family inhibits expression of the GPCR (i.e., by inhibiting one or both of transcription and translation). Hybridization can occur by conventional nucleotide complementarity to form a stable duplex, or, in the case of an anti sense nucleic acid molecule which binds with DNA duplexes, by specific interactions of the ASO with the major groove of double helical DNA encoding the GPCR. ASOs used as described herein can, for example, be administered intrathecally (e.g. by ICV or subdural injection). Alternatively, ASOs can be administered systemically. ASOs can also be delivered to cells using nucleic acid vectors (e.g., plasmids, ‘naked’ DNA vectors, conjugated or complexed {e.g. with a polyamine such as polylysine} DNA vectors, and various virus vectors) known in the art. To achieve sufficient intracellular concentrations of the antisense molecules, vector constructs in which the antisense nucleic acid molecule is placed under the control of a strong pol II or pol III promoter are preferred.

ASOs described herein can be synthesized as α-anomeric nucleic acid molecules. An α-anomeric nucleic acid molecule forms specific double-stranded hybrids with complementary RNA in which the strands run parallel to each other (Gaultier et al. (1987) Nucleic Acids Res. 15:6625-6641). The ASO can also, or instead, comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

Pharmaceutical Compositions

The invention includes pharmaceutical compositions which comprise a compound which interacts with a GPCR of at least one of the hGPR12 and hGPR03 families (e.g., an antibody which binds with the extracellular portion of such a GPCR or an ASO which modulates expression of such a GPCR). Such compositions typically comprise the compound and a pharmaceutically acceptable carrier. Supplementary active compounds can also be incorporated into the pharmaceutical compositions.

It is understood that appropriate doses of small molecule agents (e.g., small organic molecules which interact with a GPCR described herein) and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Examples of doses of a small molecule include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Examples of doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). For antibodies, examples of dosages are from about 0.1 milligram per kilogram to 100 milligrams per kilogram of body weight (generally 10 milligrams per kilogram to 20 milligrams per kilogram). If the antibody is to act in the brain, a dosage of 50 milligrams per kilogram to 100 milligrams per kilogram is usually appropriate. It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Intrathecal administration of antibodies and ASOs described herein is preferred, and such administration can be accomplished, for example, by ICV injection or by subdural injection or implantation. Solutions or suspensions used for parenteral, intradermal, intrathecal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted using acids or bases, such as hydrochloric acid or sodium hydroxide. For compositions to be administered intrathecally, use of a minimal number of components besides the active agent is preferred. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CREMOPHOR™ EL (BASF; Parsippany, N.J.) or phosphate buffered saline (PBS). The composition should be sterile and should be fluid to the extent that easy syringability exists. It should be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various anti-bacterial and anti-fungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption (e.g., aluminum monostearate or gelatin).

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, examples of methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, adjuvant materials, or both, can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™, or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as nitrogen, argon, air, or carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

Nucleic acid molecules described herein (e.g., anti-GPCR ASOs and nucleic acids encoding them) can be inserted into vectors and used as gene therapy vectors. Gene therapy vectors can be delivered to a subject by, for example, intravenous injection, intrathecal injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

It is recognized that the pharmaceutical compositions and methods described herein can be used independently or in combination with one another. That is, subjects can be administered one or more of the pharmaceutical compositions, e.g., pharmaceutical compositions comprising a nucleic acid molecule or protein of the invention or a modulator thereof, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both an agent which inhibits expression of a GPCR of one or both of the hGPR12 and hGPR03 families and an antibody which binds specifically with the extracellular portion of one of these GPCRs.

The pharmaceutical compositions can be included in a container, pack, or dispenser together with instructions for administration.

Screening Methods

The invention provides a method (also referred to herein as a “screening assay”) for identifying modulators (i.e., candidate or test compounds or agents such as peptides, peptidomimetics, small molecules or other drugs) of expression or activity (i.e., binding activity) of a GPCR of the hGPR12 or hGPR03 family. The effect of a compound (or composition) on expression or activity of a GPCR can be assessed either using a cell in which the GPCR is normally expressed or a cell which does not normally express the GPCR, but which has been transformed such that it expresses the GPCR. Alternatively, the screening method can be performed in a cell-free system in which binding of a ligand of the GPCR or binding of a G protein which normally interacts with the GPCR is assessed using a membrane- or lipid-embedded form of the GPCR or in which transcription or translation of a nucleic acid encoding the GPCR is assessed in vitro.

The screening method comprises assessing a property selected from the group consisting of:

a) transcription of a gene encoding the GPCR;

b) splicing of an RNA transcript encoding the GPCR

c) translation of an RNA transcript encoding the GPCR;

d) binding of a ligand with the extracellular portion of the GPCR;

e) binding of a G protein with the intracellular portion of the GPCR; and

f) the cytoplasmic concentration of a signal transduction mediator selected from the group consisting of Ca2+ ion, cyclic AMP, inositol 1,4,5-triphosphate, and 1,2-diacylglycerol

in the presence and absence of a test compound. A difference between the magnitude or extent of the property in the presence and absence of the test compound is an indication that the test compound is a modulator of expression or activity of the GPCR, and an indication that the test compound can be used in the methods described herein.

Test compounds which can be assessed using the screening methods can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam (1997) Anticancer Drug Des. 12:145).

Examples of methods useful for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind with the polypeptide is determined. The cell, for example, can be a yeast cell or a cell of mammalian origin. Determining the ability of the test compound to bind with the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind with the polypeptide or a biologically active portion thereof as compared to the known compound.

Therapeutic Methods

Another aspect of the invention pertains to methods of modulating expression or activity of a GPCR of either of the hGPR12 and hGPR03 families for therapeutic purposes. The modulatory method of the invention involves contacting a cell that expresses the GPCR with an agent that modulates one or more of the activities of the GPCR (e.g., an anti-GPCR ASO, an antibody, or a compound/composition identified using a screening method described herein). An example of such an agent include an ASO which inhibits (or enhances) expression of the particular GPCR (e.g., one of the ASOs described in the Examples). Another example of such an agent is an antibody which binds with the extracellular portion of the GPCR and either inhibits binding of the natural ligand thereof or substitutes for binding of the natural ligand (i.e., by binding at or near the same location, and with the same or similar effect, as the natural ligand). Still another example of such an agent is an antibody which is expressed intracellularly in a cell which normally expresses the GPCR, wherein the antibody inhibits binding between the GPCR and the G protein(s) with which it normally interacts.

It is expected that most anti-GPCR ASOs will inhibit expression of the corresponding GPCR upon administration. Nonetheless, it is understood that certain anti-GPCR ASOs can also enhance expression of the GPCR (e.g., by removing constraints on mRNA translation attributable to mRNA secondary structure).

Inhibition of expression of either of hGPR12 and hGPR03 (or a non-human mammalian ortholog thereof) can inhibit one or more of hunger, satiety, and food intake in the subject, and over time leads to reduction in body weight. Similarly, administration (particularly including intrathecal administration) of a composition that inhibits binding between the GPCR and either a normal ligand thereof or a G protein with which the GPCR normally interacts can also inhibit one or more of hunger, satiety, and food intake in the subject, and enhance eventual body weight reduction in the subject.

### EXAMPLES

The invention is now described with reference to the following Examples. These Examples are provided for the purpose of illustration only and the invention is not limited to these Examples, but rather encompass all variations which are evident as a result of the teaching provided herein.

### Example 1

### Effect of mGPR01 Antisense Oligonucleotides on Food Intake and Body Weight

The experiments presented in this Example demonstrate that inhibition of mGPR01 expression in mice using antisense oligonucleotides reduced the food intake (FI) and body weight (BW) of the mice. FI and BW were reduced both in antisense-treated mice which were permitted to feed at will and in antisense-treated mice which were forced to fast prior to being permitted to feed at will. The data presented in this example demonstrate that inhibition of expression of mGPR01 in mice, or of hGPR12 in humans, can suppress appetite and hunger.

The materials and methods used in the experiments presented in this Example are now described.

Antisense oligonucleotides (ASOs) designed using the nucleotide sequence of mGPR01 (“anti-mGPR01 ASOs”) which were used in the experiments described in the experiments presented in this Example had the designations and sequences indicated in Table I.

TABLE I

| Designation | Nucleotide Sequence | SEQ ID NO: |
|---|---|---|
| P0096 | GGGTCTTCGT TCATT | 3 |
| P0286 | GATAAGGACC ACAAC | 4 |
| P0560 | TGACATAGGT AAAGG | 5 |
| P0805 | CGATGTAGCC AGGAA | 6 |
| P0991 | GAAAGCGTAA ATGAC | 7 |

An ASO designed using the nucleotide sequence of the ob-r gene was used in these experiments, and had the nucleotide sequence 5′-GACACATCAT CTTTCTTCAG-3′ (SEQ ID NO: 8).

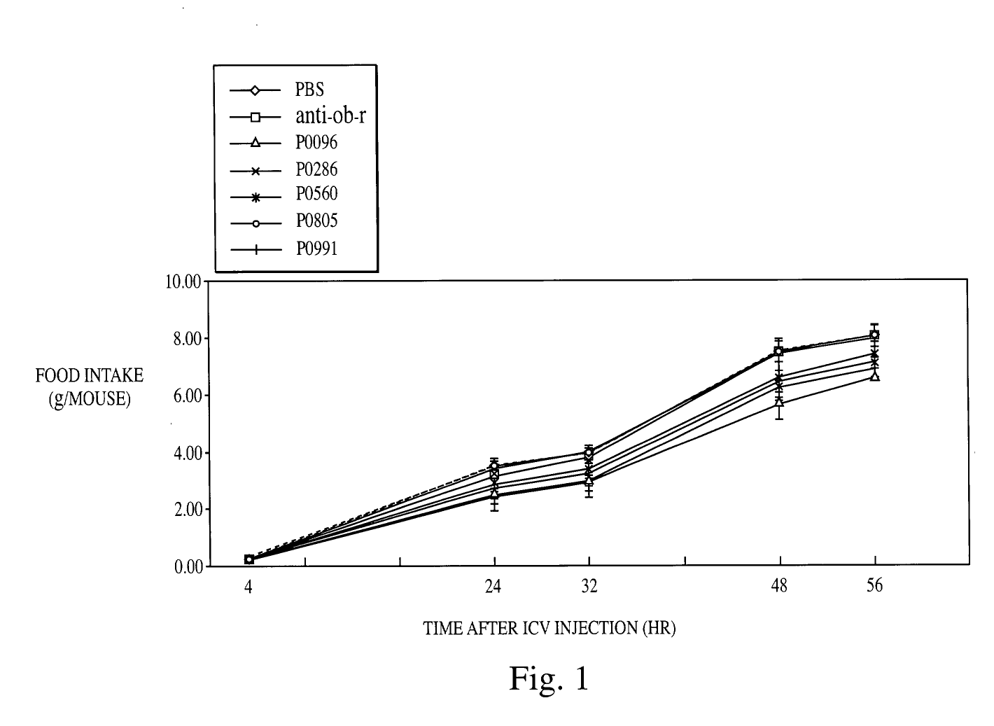

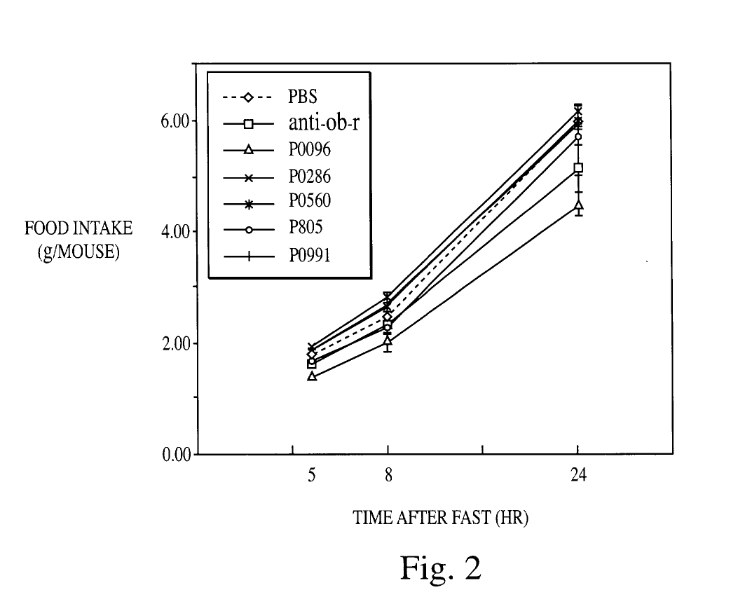

In a first set of experiments, the results of which are summarized in FIGS. 1 and 2 and Table II, ASOs (or phosphate-buffered saline {PBS} as a control) were individually administered to mice by intracerebroventricular (ICV) injection, and the effects thereof on Fl by and BW of the mice was assessed.

Male C57BL/6J mice (about 6 weeks old, obtained from the Jackson Laboratory, Bar Harbor, Me.) were individually housed in MAKROLON™ cages (Bayer; 22±2° Celsius; 12:12 hour light:dark cycle with lights off at 6 p.m.). Tap water and mouse chow were provided ad libitum except during periods of enforced fasting. Mice were stereoaxically implanted with a chronic guide cannula communicating with the cerebral third ventricle one week prior to the initiation of experiments.

Each mouse received an ICV injection on day 1 of the study. The mice in the control group received 2 microliters of PBS ICV. Mice in separate treatment groups received 0.65 nanomoles of an ASO selected from the group consisting of anti-ob-r (in 2 microliters PBS), P0096 (in 2 microliters PBS), P0286 (in 2.04 microliters PBS), P0560 (in 2.2 microliters PBS), P0805 (in 1 microliter PBS), and P0991 (in 2.04 microliters PBS). Groups of 9 (P0991) or 10 mice (all other ASOs and control) were used as individual treatment groups.

Following ICV injection, the mice were returned to their cages. BW of each mouse was determined at least daily, and FI was assessed at least twice daily. After about 56 hours, food was withheld from the mice for a period of about 16 hours, after which food was once again provided ad libitum.

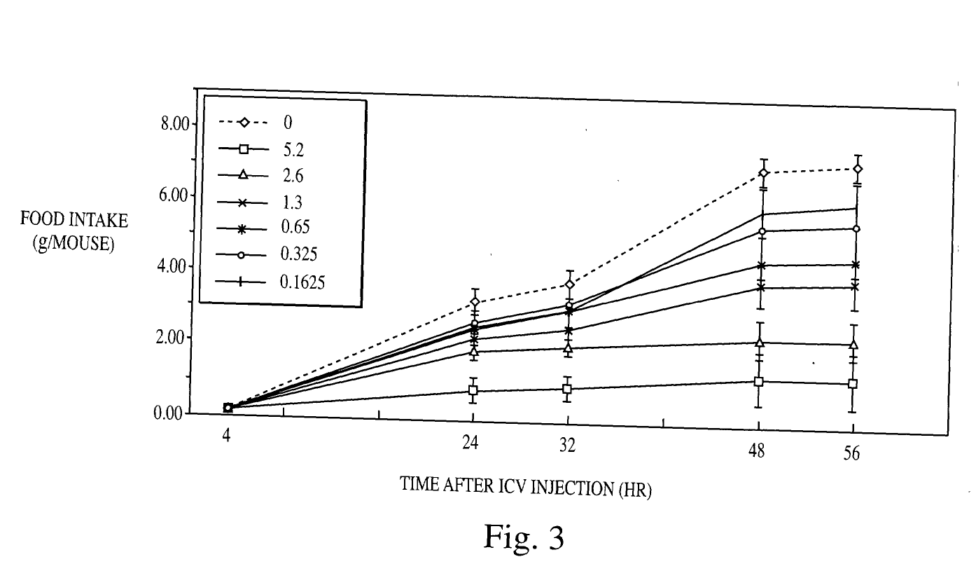

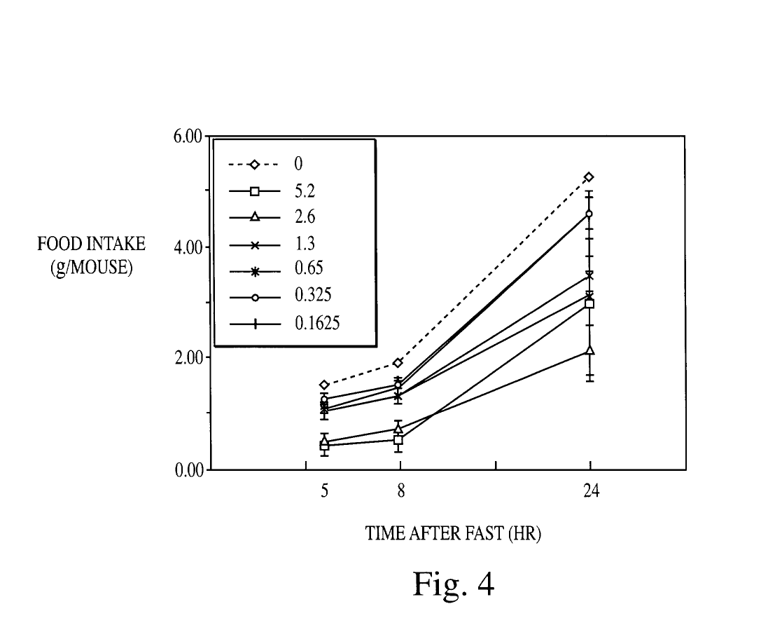

A second set of experiments (summarized in FIGS. 3 and 4 and Table III) was performed as per the first set, except that treatment groups (10 mice each) received ICV injection of only a single ASO (P0096), in selected amounts. Separate treatment groups received ICV injections containing 5.2, 2.6, 1.3, 0.65, 0.325, or 0.1625 nanomoles of P0096 per mouse. A control group received only PBS ICV (2 microliters per mouse).

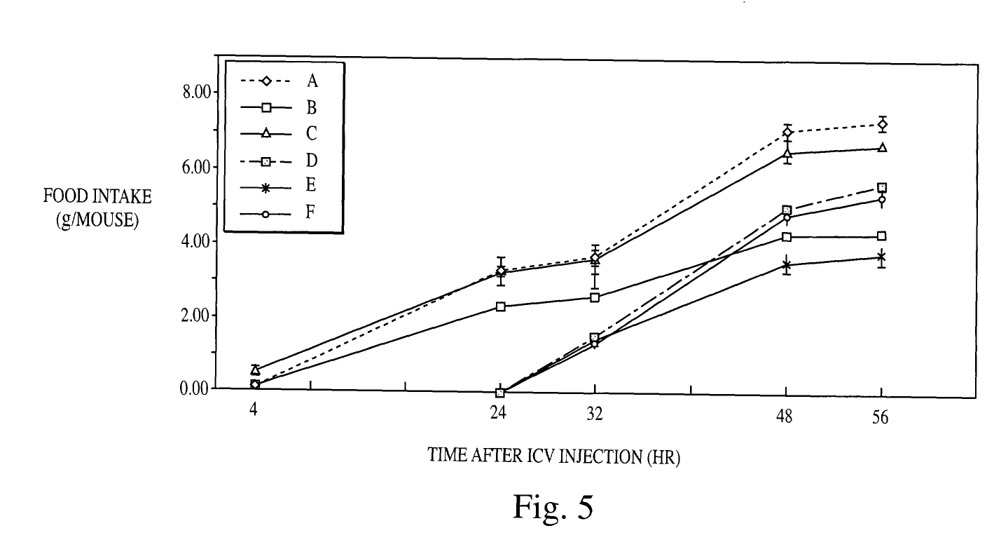

A third group of experiments (summarized in FIG. 5 and Table IV) was performed as per the first set, with the following differences. Mice were divided into six treatment groups of 10 mice each, designated A, B, C, D, E, and F. Mice in each group received an ICV injection of PBS (groups A and D; 2 microliters per mouse), P0096 (groups B and E; 0.65 nanomoles per mouse), or an oligonucleotide designated “Scramble” (groups C and F; 0.65 nanomoles per mouse). Oligonucleotide “Scramble” had the same nucleotide residue content as P0096, but in a scrambled order, and had the nucleotide sequence TGGCGTCGTC TTTAT (SEQ ID NO: 9).

Mice in groups A, B, and C were provided with food ad libitum. Mice in groups D, E, and F were forced to fast for about 24 hours following ICV injection, and were then provided with food ad libitum. FI and BW were determined at least daily for each mouse for 3 days.

The results of the experiments presented in this Example are now described.

The effects of intracerebroventricular (ICV) administration of a single 0.65 nanomole dose of selected anti-mGPR01 ASOs on cumulative FI (CFI) are summarized in FIG. 1 for mice which were permitted to feed at will for about 56 hours following ASO administration and then forced to fast for about 16 hours. The results summarized in FIG. 1 indicate that ASOs designed to exhibit expression of mGPR01 reduce CFI in mice in at least 24 hours following ICV administration. As expected, the efficacy of various ASOs varied, indicating that other anti-mGPR01 ASOs (e.g., ASOs designed and assayed by routine ‘walking’ of the mGPR01 gene or mRNA) can be designed to inhibit expression of mGPR01.

The effects of ICV administration of a single 0.65 nanomole dose of these anti-mGPR01 ASOs on CFI are summarized in FIG. 2 for mice following a forced 16 hour fast (i.e., a fast imposed following 56 hours of eating at will). These results indicate that anti-mGPR01 ASOs can inhibit not only the basal level of FI, but can also inhibit hunger induced by fasting.

The average initial body weight (BW) and BW of mice on day 5 of the experiments summarized in FIGS. 1 and 2 are presented in Table II. These results indicate that ICV administration of anti-mGPR01 ASOs can lead to reduction of BW over time.

TABLE II

| | |
|---|---|
| Composition | Body Weight (grams; mean ± SEM) |

| Administered | On Day 1 | On Day 5 |
|---|---|---|
| PBS | 24.02 ± 0.47 | 24.05 ± 0.47 |
| anti-ob-r | 23.92 ± 0.46 | 22.67 ± 0.40* |
| P0096 | 24.06 ± 0.48 | 21.62 ± 0.61** |
| P0286 | 24.05 ± 0.46 | 23.36 ± 0.48 |
| P0560 | 24.06 ± 0.45 | 23.67 ± 0.55 |
| P0805 | 24.07 ± 0.50 | 23.73 ± 0.62 |
| P0991 | 23.96 ± 0.40 | 23.54 ± 0.44 |

*p < 0.05 versus PBS control group
**p < 0.01 versus PBS control group

The results summarized in FIGS. 3 and 4 demonstrate that a dose-response relationship exists between the amount of P0096 administered ICV and the resulting reduction in CFI. This dose-response relationship exists both during ad libitum feeding and following enforced fasting, and indicates that the reduction in CFI is attributable to physiological effects mediated by ICV injection of P0096. The results summarized in Table III (comparing initial BW and BW following treatment) further confirm a dose-response relationship between P0096 administration and reduction of BW.

TABLE III

| | |
|---|---|
| P0096 dose | Body Weight (grams; mean ± SEM) |

| (nanograms per mouse) | On Day 1 | On Day 5 |
|---|---|---|
| 0 (PBS) | 23.85 ± 0.41 | 24.13 ± 0.29 |
| 5.2 | 23.61 ± 0.50 | 18.63 ± 3.36¹** |
| 2.6 | 23.76 ± 0.50 | 16.96 ± 0.90²** |
| 1.3 | 23.90 ± 0.52 | 19.59 ± 0.81** |
| 0.65 | 24.02 ± 0.55 | 20.52 ± 0.78** |
| 0.325 | 23.72 ± 0.41 | 22.77 ± 0.56* |
| 0.1625 | 23.78 ± 0.40 | 22.77 ± 0.72 |

Notes:
¹Average of the 3 mice which survived to day 5
²Average of the 9 mice which survived to day 5
*p < 0.05 versus PBS control groups
**p < 0.01 versus PBS control groups

The results summarized in FIG. 5 demonstrate that the effect of ICV injection of P0096 on CFI depended the particular sequence (i.e., SEQ ID NO: 3) of P0096. The results presented in Table IV (comparing initial BW and BW following administration of PBS, P0096, or Scramble) demonstrate that the effect of ICV injection of P0096 on BW were also attributable to the sequence of P0096.

TABLE IV

| Composition | | Body Weight (grams; mean ± SEM) | |
|---|---|---|---|
| Administered | Food¹ | On Day 1 | On Day 3 |
| PBS | +/+ | 22.25 ± 0.25 | 23.13 ± 0.29 |
| P0096 (0.65 nanomoles | +/+ | 22.85 ± 0.07 | 21.22 ± 0.20** |
| per mouse) | | | |
| Scramble (0.65 | +/+ | 22.75 ± 0.40 | 22.01 ± 0.26* |
| nanomoles per mouse) | | | |
| PBS | −/+ | 23.35 ± 0.08 | 23.53 ± 0.30 |
| P0096 (0.65 nanomoles | −/+ | 24.37 ± 0.08 | 22.70 ± 0.37 |
| per mouse) | | | |
| Scramble (0.65 | −/+ | 23.20 ± 0.46 | 22.88 ± 0.52 |
| nanomoles per mouse) | | | |

Notes:
¹+/+ indicates food provided ad libitum throughout the experiment; −/+indicates that the mice were forced to fast for 24 hours following ICV injection, and were then provided with food ad libitum for the remainder of the experiment.
*p < 0.05 versus PBS control groups
**p <0.01 versus PBS control groups

### Example 2

### Effect of mGPR21 Antisense Oligonucleotides on Food Intake and Body Weight

The experiments presented in this Example demonstrate that inhibition of mGPR21 expression in mice using antisense oligonucleotides reduced the food intake (FI) and body weight (BW) of the mice. FI and BW were reduced both in antisense-treated mice which were permitted to feed at will and in antisense-treated mice which were forced to fast prior to being permitted to feed at will. The data presented in this example demonstrate that inhibition of expression of mGPR21 in mice, or of hGPR03 in humans, can suppress appetite and hunger.

The materials and methods used in the experiments presented in this Example are now described.

Antisense oligonucleotides (ASOs) designed using the nucleotide sequence of mGPR21 (“anti-mGPR21 ASOs”) which were used in the experiments described in the experiments presented in this Example had the designations and sequences indicated in Table V.

TABLE V

| Designation | Nucleotide Sequence | SEQ ID NO: |
|---|---|---|
| P0519 | AACCAGGCCA TAGAG | 13 |
| P0973 | ACCCACCCAC ACCAA | 14 |
| P1080 | GCCAGAACCA CCAGA | 15 |
| P1332 | AGGGTAAGAT AGGTG | 16 |
| P1478 | ACTAGACATC ACTAG | 17 |

An ASO designed using the nucleotide sequence of the ob-r gene was used in these experiments, and had the nucleotide sequence GACACATCAT CTTTCTTCAG (SEQ ID NO: 8).

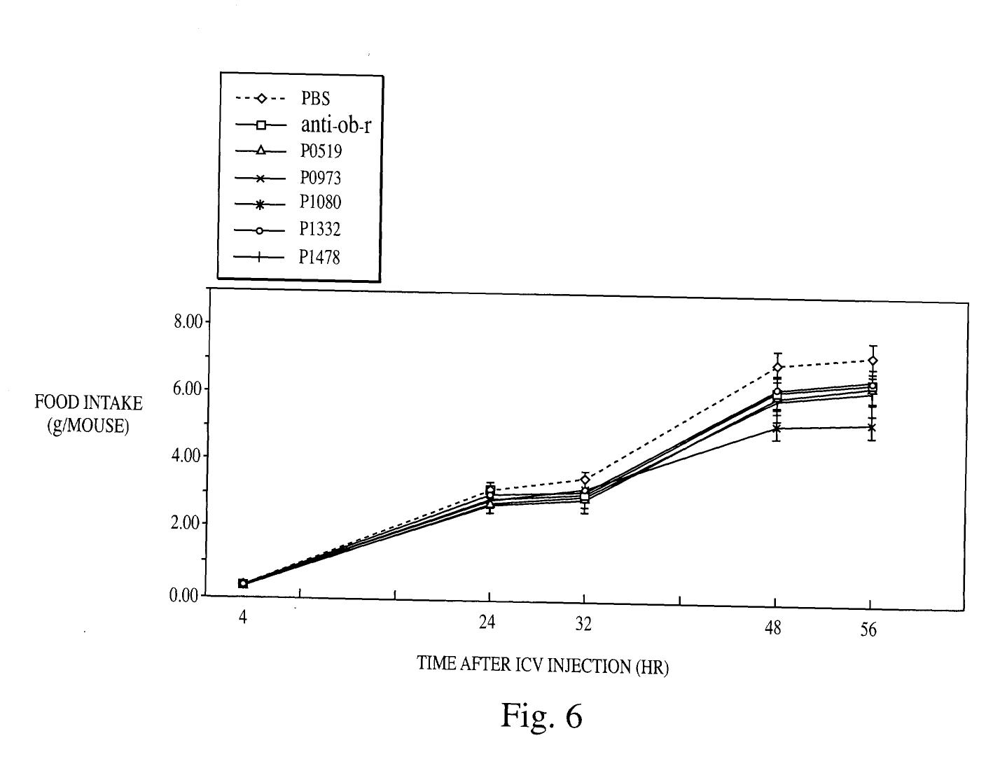

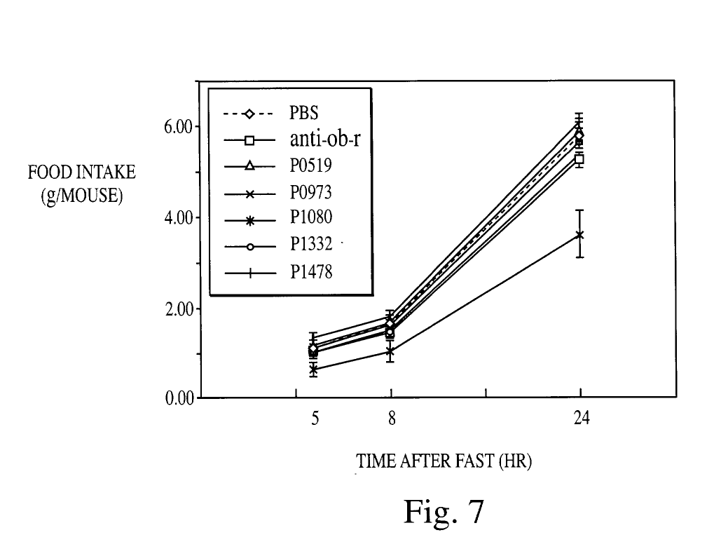

In a first set of experiments, the results of which are summarized in FIGS. 6 and 7 and Table VI, ASOs (or phosphate-buffered saline {PBS} as a control) were individually administered to mice by intracerebroventricular (ICV) injection, and the effects thereof on FI by and BW of the mice was assessed.

Male C57BL/6J mice (about 6 weeks old, obtained from the Jackson Laboratory, Bar Harbor, Me.) were individually housed in MAKROLON™ cages (Bayer; 22±2° Celsius; 12:12 hour light:dark cycle with lights off at 6 p.m.). Tap water and mouse chow were provided ad libitum except during periods of enforced fasting. Mice were stereoaxically implanted with a chronic guide cannula communicating with the cerebral third ventricle one week prior to the initiation of experiments.

Each mouse received an ICV injection on day 1 of the study. The mice in the control group received 2 microliters of PBS ICV. Mice in separate treatment groups received 0.65 nanomoles of an ASO selected from the group consisting of anti-ob-r (in 2 microliters PBS), P0519 (in 2 microliters PBS), P0973 (in 1.71 microliters PBS), P1080 (in 2.7 microliters PBS), P1332 (in 2.2 microliters PBS), and P1478 (in 3.1 microliters PBS). Groups of or 10 mice were used as individual treatment groups.

Following ICV injection, the mice were returned to their cages. BW of each mouse was determined at least daily, and FI was assessed at least twice daily. After about 56 hours, food was withheld from the mice for a period of about 16 hours, after which food was once again provided ad libitum.

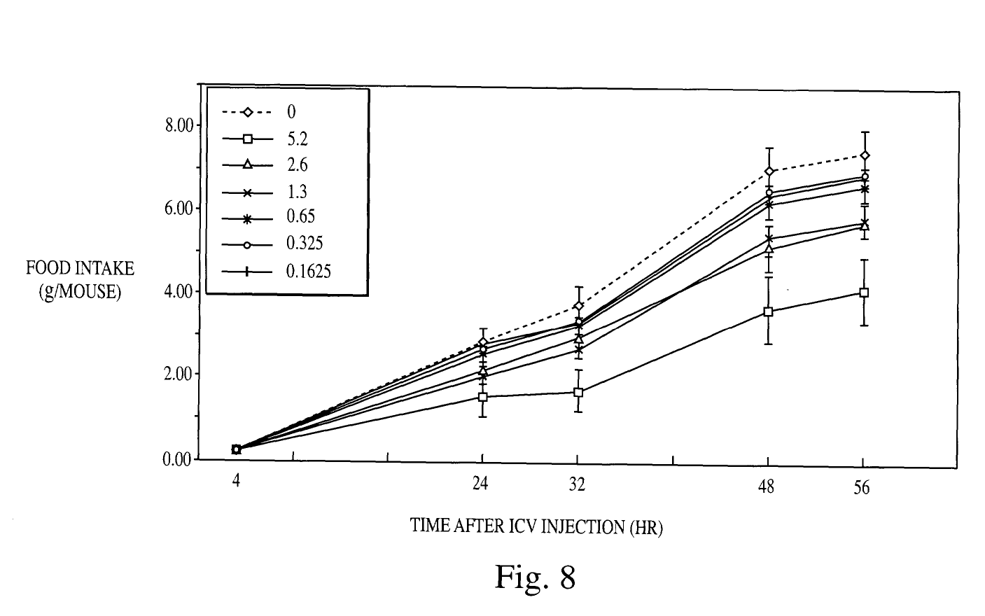

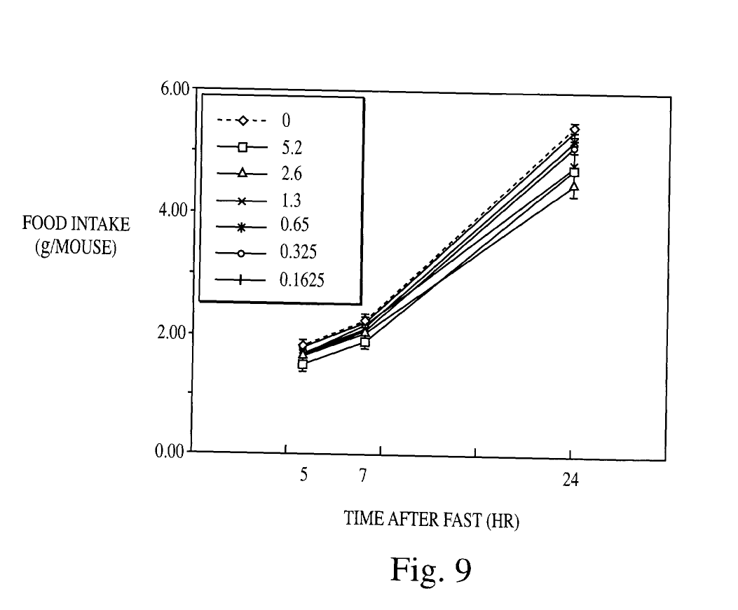

A second set of experiments (summarized in FIGS. 8 and 9 and Table VII) was performed as per the first set, except that treatment groups (10 mice each) received ICV injection of only a single ASO (P1080), in selected amounts. Separate treatment groups received ICV injections containing 5.2, 2.6, 1.3, 0.65, 0.325, or 0.1625 nanomoles of P1080 per mouse. A control group received only PBS ICV (2 microliters per mouse).

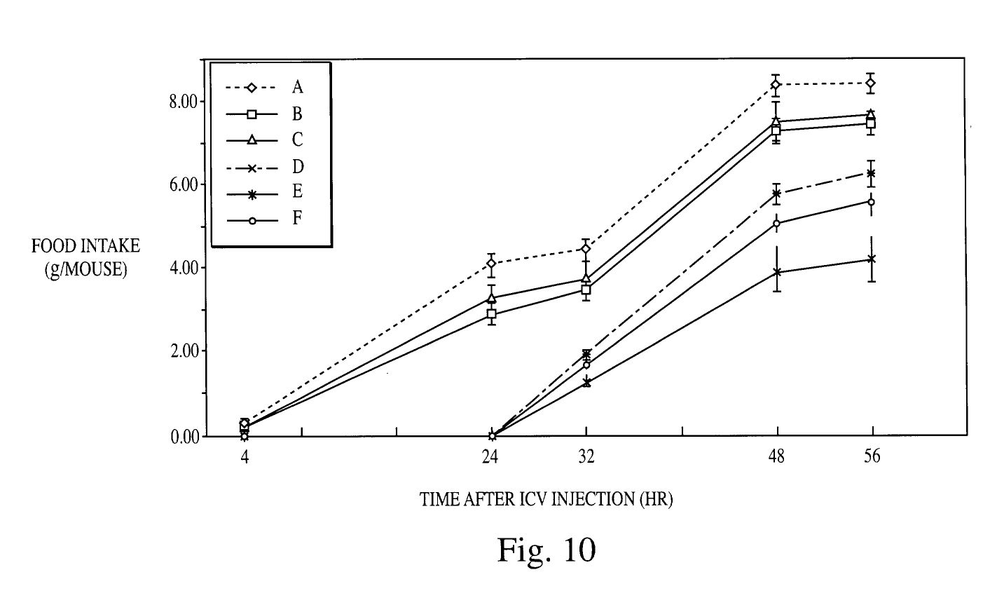

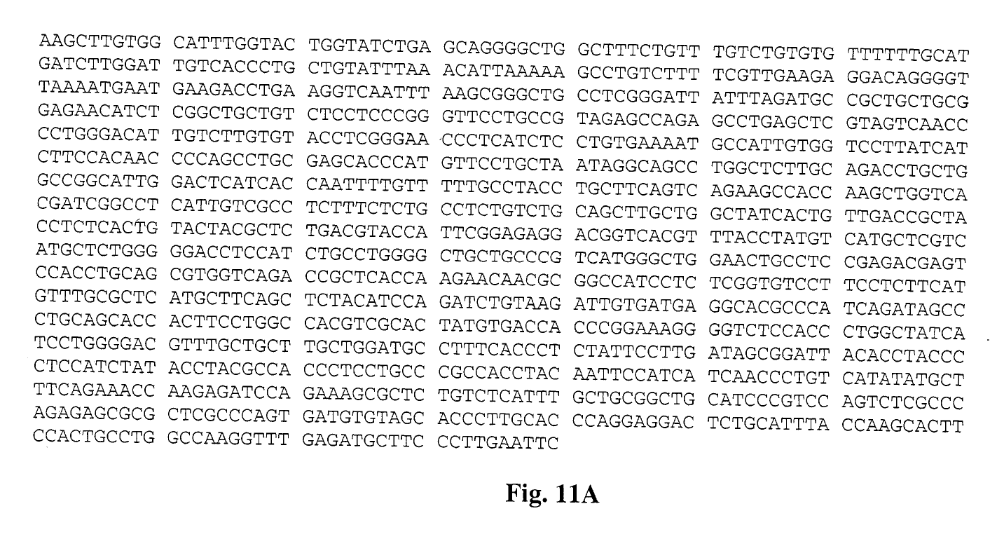

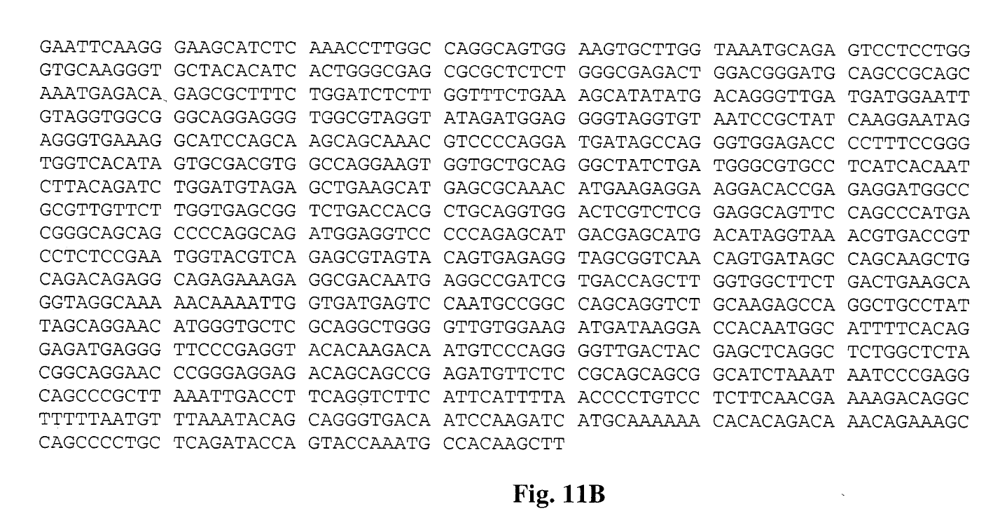

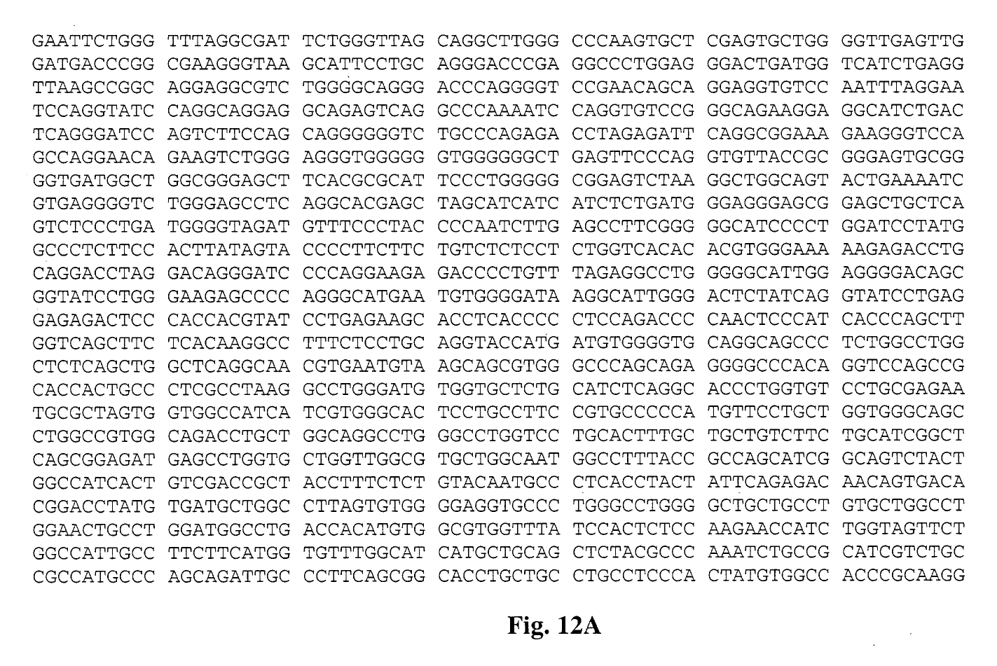

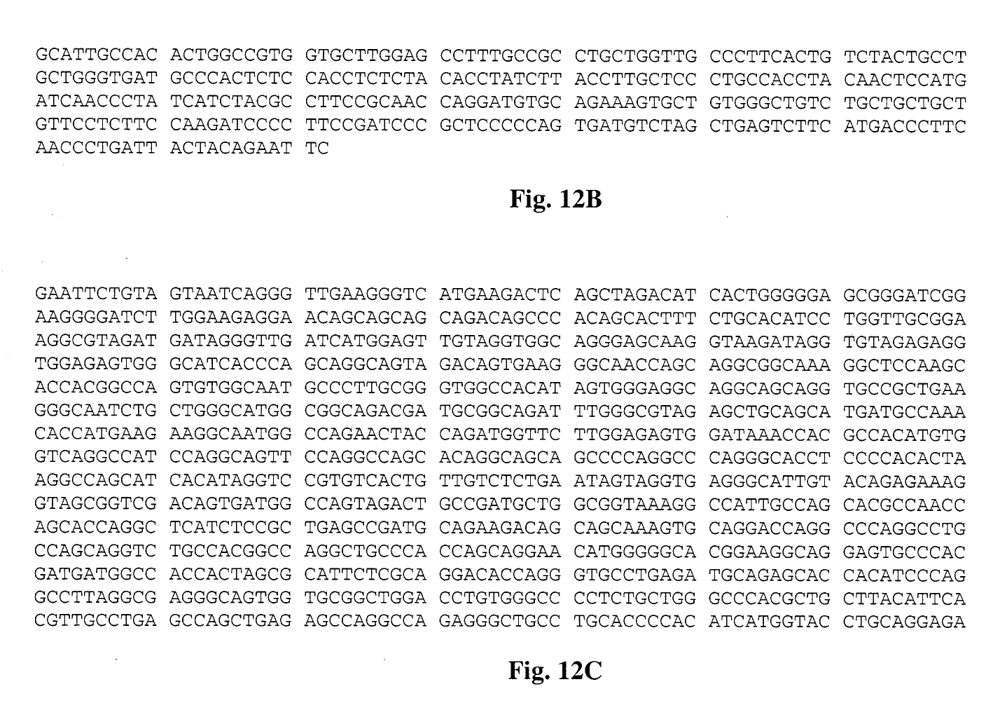

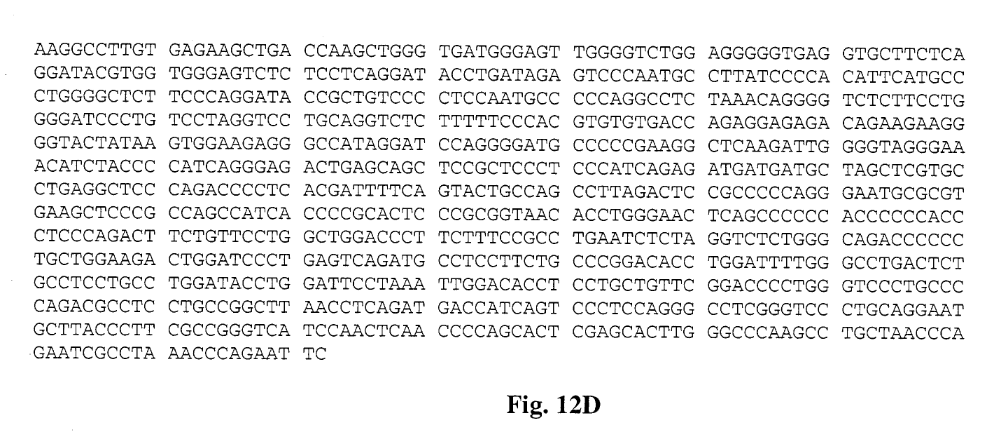

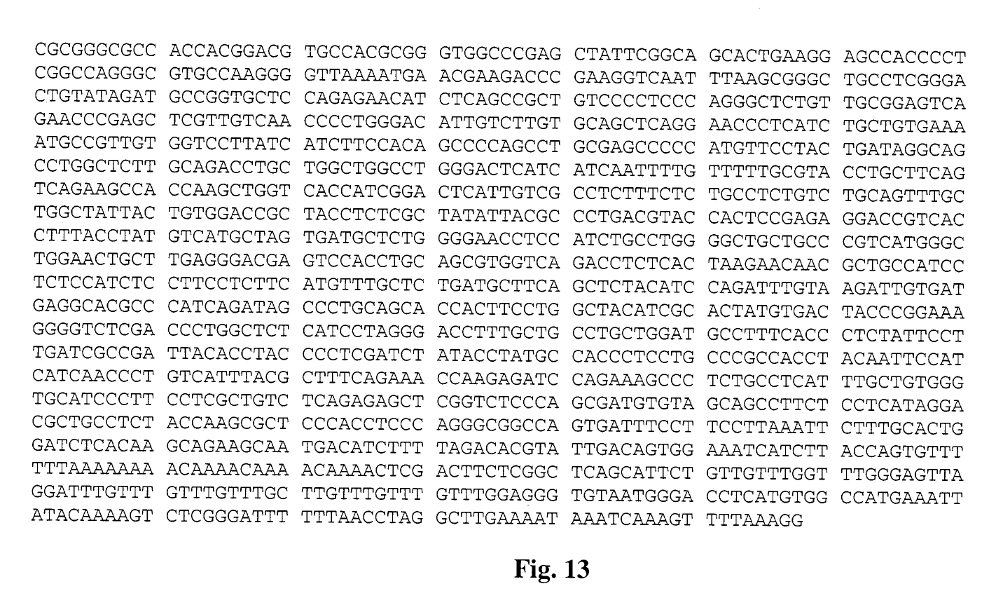

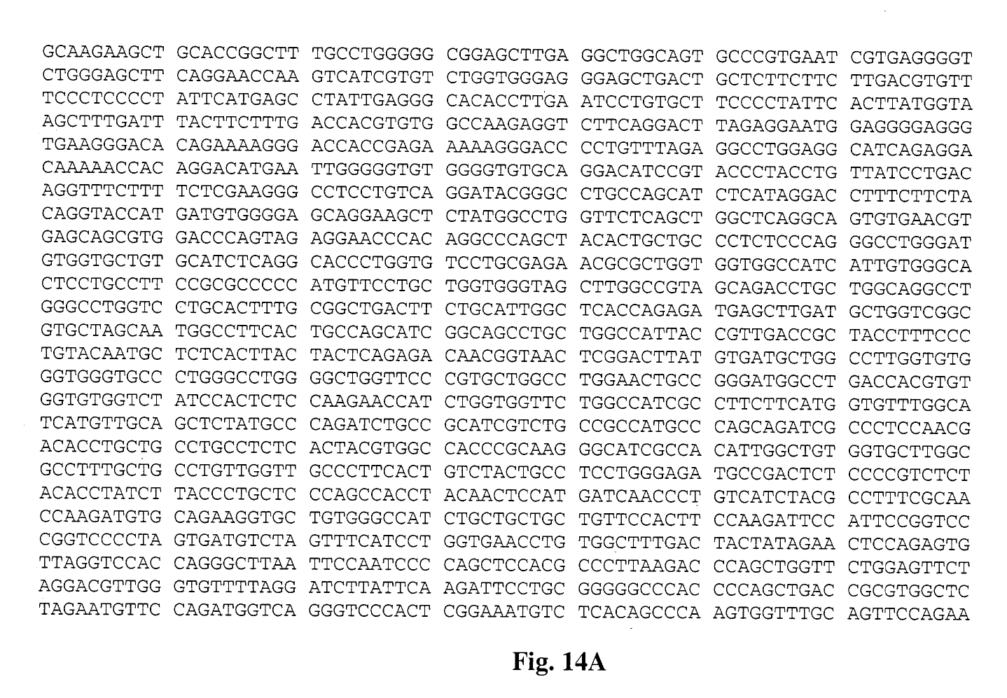

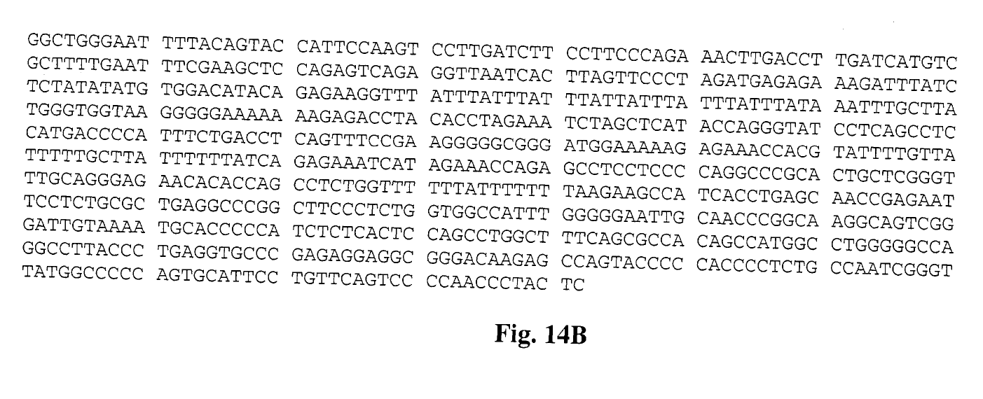

A third group of experiments (summarized in FIG. 10 and Table VIII) was performed as per the first set, with the following differences. Mice were divided into six treatment groups of 10 mice each, designated A, B, C, D, E, and F. Mice in each group received an ICV injection of PBS (groups A and D; 2 microliters per mouse), P1080 (groups B and E; 0.65 nanomoles per mouse), or an oligonucleotide designated “Sense” (groups C and F; 0.65 nanomoles per mouse). Oligonucleotide “Sense” was the sense strand corresponding to P1080 (i.e., the complement of P1080), and had the nucleotide sequence TCTGGTGGTT CTGGC (SEQ ID NO: 19).

Mice in groups A, B, and C were provided with food ad libitum. Mice in groups D, E, and F were forced to fast for about 24 hours following ICV injection, and were then provided with food ad libitum. FI and BW were determined at least daily for each mouse for 3 days.

The results of the experiments presented in this Example are now described.

The effects of ICV administration of a single 0.65 nanomole dose of selected anti-mGPR21 ASOs on (CFI) are summarized in FIG. 6 for mice which were permitted to feed at will for about 56 hours following ASO administration and then forced to fast for about 16 hours. The results summarized in FIG. 6 indicate that ASOs designed to exhibit expression of mGPR21 reduce CFI in mice in at least 24 hours following ICV administration. As expected, the efficacy of various ASOs varied, indicating that other anti-mGPR21 ASOs (e.g., ASOs designed and assayed by routine ‘walking’ of the mGPR21 gene or mRNA) can be designed to inhibit expression of mGPR21.

The effects of ICV administration of a single 0.65 nanomole dose of these anti-mGPR21 ASOs on CFI are summarized in FIG. 7 for mice following a forced 16 hour fast (i.e., a fast imposed following 56 hours of eating at will). These results indicate that anti-mGPR21 ASOs can inhibit not only the basal level of FI, but can also inhibit hunger induced by fasting.

The average initial body weight (BW) and BW of mice on day 5 of the experiments summarized in FIGS. 6 and 7 are presented in Table VI. These results indicate that ICV administration of anti-mGPR21 ASOs can lead to reduction of BW over time.

TABLE VI

| | |
|---|---|
| Composition | Body Weight (grams; mean ± SEM) |

| Administered | On Day 1 | On Day 5 |
|---|---|---|
| PBS | 25.49 ± 0.47 | 25.46 ± 0.40 |
| anti-ob-r | 25.38 ± 0.62 | 24.80 ± 0.40 |
| P0519 | 25.45 ± 0.61 | 25.89 ± 0.46 |
| P0973 | 25.36 ± 0.51 | 25.03 ± 0.45 |
| P1080 | 25.47 ± 0.47 | 22.44 ± 0.49** |
| P1332 | 25.54 ± 0.45 | 25.29 ± 0.39 |
| P1478 | 25.13 ± 0.86 | 25.62 ± 0.38 |

Note:
**p < 0.01 versus PBS control groups

The results summarized in FIGS. 8 and 9 demonstrate that a dose-response relationship exists between the amount of P1080 administered ICV and the resulting reduction in CFI. This dose-response relationship exists both during ad libitum feeding and following enforced fasting, and indicates that the reduction in CFI is attributable to physiological effects mediated by ICV injection of P1080. The results summarized in Table VII (comparing initial BW and BW following treatment) further confirm a dose-response relationship between P1080 administration and reduction of BW. It may be that the dose-response relationship between P1080 administration and BW reduction is more pronounced for mice feeding at will (i.e., compare BW values at days 1 and 3) than for mice feeding after being forced to fast (i.e., compare BW values at days 3 and 5). This suggests that inhibition of mGPR21 expression (e.g., effected by administration of an anti-mGPR21 ASO) can modulate sensation of satiety during normal food ingestion differently than it modulates sensation of hunger during fasting. Thus, anti-mGPR21 ASOs can be used to modulate timing of food ingestion by the mammal without significantly affecting the amount of food ingested by a mammal once the mammal senses hunger. Alternatively, anti-mGPR21 ASOs can be used to modulate the amount of food ingested by a mammal once the mammal has chosen to dine without significantly affecting the timing of hunger sensation by the mammal.

TABLE VII

| P1080 dose | | | |
|---|---|---|---|
| (nanograms | Body Weight (grams; mean ± SEM) | | |
| per mouse) | On Day 1 | On Day 3 | On Day 5 |
| 0 (PBS) | 24.62 ± 0.64 | 25.52 ± 0.37 | 25.58 ± 0.43 |
| 5.2 | 25.21 ± 0.44 | 22.29 ± 0.80** | 23.10 ± 0.73** |
| 2.6 | 25.31 ± 0.42 | 23.17 ± 0.76* | 23.82 ± 0.71* |
| 1.3 | 25.36 ± 0.42 | 24.88 ± 0.38 | 24.84 ± 0.63 |
| 0.65 | 25.48 ± 0.42 | 25.57 ± 0.49 | 25.97 ± 0.52 |
| 0.325 | 25.25 ± 0.37 | 25.65 ± 0.47 | 25.79 ± 0.43 |
| 0.1625 | 25.34 ± 0.36 | 25.60 ± 0.35 | 25.80 ± 0.29 |

Notes:
*p < 0.05 versus PBS control groups
**p < 0.01 versus PBS control groups

The results summarized in FIG. 10 demonstrate that the effect of ICV injection of P1080 on CFI depended the particular sequence (i.e., SEQ ID NO: 15) of P1080. The results presented in Table VIII (comparing initial BW and BW following administration of PBS, P1080, or the oligonucleotide designated “Sense”) demonstrate that the effect of ICV injection of P1080 on BW were also attributable to the sequence of P1080.

TABLE VIII

| Composition | | Body Weight (grams; mean ± SEM) | |
|---|---|---|---|
| Administered | Food¹ | On Day 1 | On Day 3 |
| PBS | +/+ | 23.84 ± 0.47 | 24.79 ± 0.49 |
| P 1080 (0.65 nanomoles | +/+ | 24.32 ± 0.57 | 24.57 ± 0.55 |
| per mouse) | | | |
| Sense (0.65 nanomoles | +/+ | 24.37 ± 0.57 | 24.37 ± 0.61 |
| per mouse) | | | |
| PBS | −/+ | 24.47 ± 0.61 | 24.68 ± 0.55 |
| P 1080 (0.65 nanomoles | −/+ | 24.55 ± 0.61 | 23.08 ± 0.67 |
| per mouse) | | | |
| Sense (0.65 nanomoles | −/+ | 24.74 ± 0.70 | 24.19 ± 0.71 |
| per mouse) | | | |

Note:
¹+/+ indicates food provided ad libitum throughout the experiment; −/+ indicates that the mice were forced to fast for 24 hours following ICV injection, and were then provided with food ad libitum for the remainder of the experiment.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A composition for suppressing hunger in a mammal, the composition comprising an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03.

2. The composition of claim 1, wherein the mammal is a human and the polynucleotide is selected from the group consisting of the sense strand of a cDNA encoding hGPR12 and the sense strand of a cDNA encoding hGPR03.

3. The composition of claim 2, wherein the polynucleotide is a DNA molecule having the sequence SEQ ID NO: 2.

4. The composition of claim 2, wherein the polynucleotide is a DNA molecule having the sequence SEQ ID NO: 12.

5. The composition of claim 1, wherein the oligonucleotide comprises 15 nucleotide residues.

6. The composition of claim 1, wherein the oligonucleotide has a nucleotide sequence selected from the group consisting of SEQ ID NOs: 3-7 and 13-17.

7. The composition of claim 1, wherein the composition further comprises a pharmaceutically acceptable carrier.

8. The composition of claim 1, wherein the composition is formulated for intrathecal administration.

9. The composition of claim 1, wherein the composition is formulated for sustained release of the oligonucleotide.

10. A method of suppressing hunger in a mammal, the method comprising intrathecally administering to the mammal an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03.

11. A composition for enhancing satiety in a mammal, the composition comprising an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03.

12. A method of enhancing satiety in a mammal, the method comprising intrathecally administering to the mammal an oligonucleotide which hybridizes under stringent conditions with a polynucleotide selected from the group consisting of the sense strand of a cDNA encoding hGPR12, the sense strand of a cDNA encoding hGPR03, the sense strand of a cDNA encoding the mammal's ortholog of hGPR12, and the sense strand of a cDNA encoding the mammal's ortholog of hGPR03.

13. A method of assessing whether a composition is a modulator of hunger in a mammal, the method comprising comparing
a) the change in the cytoplasmic concentration of a signal transduction mediator selected from the group consisting of Ca²⁺ ion, cyclic AMP, inositol 1,4,5-triphosphate, and 1,2-diacylglycerol attributable to the presence of the composition in a medium contacting a first cell which comprises a G protein-coupled receptor protein selected from the group consisting of hGPR12, hGPR03, the mammal's ortholog of hGPR12, and the mammal's ortholog of hGPR03; and
b) the change in the cytoplasmic concentration of the same signal transduction mediator attributable to the presence of the composition in a medium contacting a second cell of the same type which does not comprise the G protein-coupled receptor protein;
wherein a difference between
i) the change attributable to the presence of the composition in a medium contacting the first cell and
ii) the change attributable to the presence of the composition in a medium contacting the second cell
is an indication that the composition is a modulator of hunger in the mammal.

14. A method of assessing whether a composition is a modulator of satiety in a mammal, the method comprising comparing
a) the change in the cytoplasmic concentration of a signal transduction mediator selected from the group consisting of Ca2+ ion, cyclic AMP, inositol 1,4,5-triphosphate, and 1,2-diacylglycerol attributable to the presence of the composition in a medium contacting a first cell which comprises a G protein-coupled receptor protein selected from the group consisting of hGPR12, hGPR03, the mammal's ortholog of hGPR12, and the mammal's ortholog of hGPR03; and
b) the change in the cytoplasmic concentration of the same signal transduction mediator attributable to the presence of the composition in a medium contacting a second cell of the same type which does not comprise the G protein-coupled receptor protein;
wherein a difference between
i) the change attributable to the presence of the composition in a medium contacting the first cell and
ii) the change attributable to the presence of the composition in a medium contacting the second cell
is an indication that the composition is a modulator of hunger in the mammal.

15. An antisense oligonucleotide consisting of from 15 to 300 nucleotide residues, wherein the nucleotide sequence of the oligonucleotide is at least 75% identical with a portion of one of SEQ ID NOs: 20 and 21, as assessed using the NBLAST program with score=100, wordlength=12, gap opening penalty=11, gap extension penalty=1, and lambda ratio=0.85.

16. An antisense oligonucleotide consisting of from 15 to 300 nucleotide residues, wherein the nucleotide sequence of the oligonucleotide is at least 75% identical with a portion of one of SEQ ID NOs: 20 and 21, as assessed using the ALIGN program with PAM120 weight residue table, gap opening penalty=11, gap extension penalty=1, and lambda ratio=0.85.

17. An antisense oligonucleotide consisting of from 15 to 300 nucleotide residues, wherein the nucleotide sequence of the oligonucleotide is at least 75% identical with a portion of one of SEQ ID NOs: 20 and 21, as assessed by non-gapped sequence alignment.

18. A method of modulating food intake in a mammal, the method comprising intrathecally administering to the mammal an antibody raised against an extracellular portion of a protein selected from the group consisting of hGPR12, hGPR03, the mammal's ortholog of hGPR12, and the mammal's ortholog of hGPR03.

19. A method of making an antibody for modulating food intake in a mammal, the method comprising
administering to an immunocompetent vertebrate a membrane-embedded protein selected from the group consisting of hGPR12, hGPR03, the mammal's ortholog of hGPR12, and the mammal's ortholog of hGPR03; and thereafter
recovering an antibody which binds specifically with the protein from the vertebrate's serum, whereby the antibody is useful for modulating food intake in a mammal.

20. A method of making an antibody for modulating food intake in a mammal, the method comprising
administering to an immunocompetent vertebrate a membrane-embedded protein selected from the group consisting of hGPR12, hGPR03, the mammal's ortholog of hGPR12, and the mammal's ortholog of hGPR03; thereafter
isolating an antibody-producing cell which produces an antibody which binds specifically with the protein from the vertebrate;
forming a hybridoma using the antibody-producing cell; and
recovering the antibody from a medium in which the hybridoma is maintained, whereby the antibody is useful for modulating food intake in a mammal.
